(19) United States
(12) Patent Application Publication
(10) Pub. No.: US 2011/0190265 A1
(43) Pub. Date: Aug. 4, 2011

(54) METHODS AND COMPOSITIONS FOR TREATING BACTERIAL INFECTIONS BY INHIBITING QUORUM SENSING

(76) Inventor: Vern L. Schramm, New York, NY (US)

(21) Appl. No.: 12/998,129

(22) PCT Filed: Sep. 18, 2009
(86) PCT No.: PCT/US2009/005229

§ 371 Date: Apr. 18, 2011

Related U.S. Application Data
(60) Provisional application No. 61/192,796, filed on Sep. 22, 2008.

(51) Int. Cl. A61K 31/519; A61P 31/04

(52) U.S. Cl. 514/210.21; 514/265.1

(57) ABSTRACT

The present invention provides methods for treating bacterial infections in a subject comprising administering to the subject a sub-growth inhibiting amount of a 5′-Methylthioadenosine/S-adenosyl homocysteine nucleosidase (MTAN) inhibitor. The present invention further provides pharmaceutical compositions comprising a sub-bacterial-growth inhibiting amount of a 5′-Methylthioadenosine/S-adenosyl homocysteine nucleosidase (MTAN) inhibitor and a pharmaceutically acceptable carrier.

### CROSS-REFERENCE TO RELATED APPLICATION

This application claims priority of U.S. Provisional Patent Application No. 61/192,796, filed on Sep. 22, 2008, the content of which is incorporated by reference.

### STATEMENT OF GOVERNMENT SUPPORT

This invention was made with U.S. Government support under Grant No. GM41916 from the National Institutes of Health. The U.S. Government has certain rights in the invention.

### FIELD OF THE INVENTION

The present invention relates to methods and compositions for treating bacterial infections by inhibiting quorum sensing in the bacteria.

### BACKGROUND OF THE INVENTION

Throughout this application various publications are referred to by Arabic numerals in superscript. Full citations for these references may be found at the end of the specification immediately preceding the claims. The disclosures of these publications are hereby incorporated by reference in their entireties into the subject application to more fully describe the art to which the subject application pertains.

Bacteria communicate to each other by a process known as quorum sensing. When the population density reaches critical levels, they produce and detect signaling molecules known as autoinducers (AIs) or pheromones to coordinate gene expression and regulate processes beneficial to the microbial communities¹. Many bacterial species use quorum sensing to regulate virulence²⁻⁶. Several mutant bacterial strains defective in quorum sensing create less potent infections. Quorum sensing-deficient intranasal Streptococcus pneumoniae infections in the mouse are less effective at spreading to the lungs or the bloodstream⁷. In an infant rat Neisseria meningitidis infection model, a quorum sensing-deficient strain is unable to produce viable bacteria in the blood⁸. Furthermore, rats infected with a quorum sensing-deficient Pseudomonas aeruginosa mutant strain had significantly lower bronchial and pulmonic bacterial counts⁹.

5′-Methylthioadenosine/S-adenosyl homocysteine nucleosidases (MTANs) play a crucial role in maintaining homeostasis in bacteria. MTANs are tightly involved in the utilization of S-adenosyl methionine (SAM), towards methylation reactions yielding S-adenosyl homocysteine (SAH), and polyamine biosynthesis producing methylthioadenosine (MTA) (FIG. 1). MTANs catalyze the irreversible hydrolytic deadenylation of MTA and SAH (FIG. 2). MTANs are the only known route for SAH and MTA metabolism in bacteria, whose accumulation is expected to inhibit related pathways. In addition, MTANs are directly involved in the biosynthesis of autoinducers, used by bacteria in quorum sensing. AI-1 and AI-2 are two classes of autoinducers synthesized from SAM, and MTAN is central to their biosyntheses (FIG. 1). AI-1 is a family of acyl-homoserine lactones (AHLs) and is believed to provide signaling molecules for intra-species communication. In the synthesis of AHLs, SAM produces MTA as by-product, and MTAN provides the only known means to metabolize MTA in bacteria. AI-2 includes derivatives of 4,5-dihydroxy-2,3-pentanedione (DPD), responsible for inter-species communication. MTAN produces S-ribosylhomocysteine (SRH) from SAH, and SRH is converted by LuxS to homocysteine and DPD, which undergoes cyclization and hydrolysis to produce AI-2s (FIG. 1). Blocking MTAN activity is expected to cause accumulation of MTA, resulting in product inhibition of AI-1 production by AHL synthase¹⁰. In addition, inhibition of MTAN can directly block the formation of SRH, the precursor of AI-2.

Human MTAP or 5′-methylthioadenosine phosphorylase is MTAN's counterpart in humans, and functions similarly in metabolizing MTA but uses phosphate as a nucleophile instead of water. It has been identified as an anticancer target due to its involvement in polyamine biosynthesis and purine salvage pathways^{11,12}. The transition state structures of human MTAP as well as MTANs from Escherichia coli (EcMTAN), Streptococcus pneumoniae (SpMTAN), and Neisseria meningitidis (NmMTAN) have been solved using kinetic isotope effects¹³⁻¹⁶. They all have dissociative S_{N}1 transition states with ribooxacarbenium ion character, but while human MTAP, EcMTAN, and SpMTAN all have a “late’ transition state with a fully broken N-glycosidic bond (i.e., C1′-N9 distance of 3.0 Å or greater), NmMTAN has an “early” transition state and a C1′-N9 distance of 1.68 Å (FIG. 2). The human MTAP transition state differs from those of the MTANs in the significant participation of the phosphate nucleophile, whereas the water nucleophile in the bacterial enzymes does not participate in bond formation at the transition state. Another key difference in the transition states is the adenine N7 protonation state, and hence, the overall charge of the leaving group. Human MTAP and SpMTAN transition states both have their adenine N7 unprotonated and anionic. In both EcMTAN and NmMTAN, N7 is protonated, resulting in a leaving group that is neutral for the former, and cationic for the latter (due to significant bond order to the C1′-N9 bond at the transition state).

Transition state analogue design in the study of purine nucleoside phosphorylases (PNPs) has yielded extremely potent inhibitors currently in clinical trials for autoimmune disease and cancer¹⁷⁻²⁰, and the same drug design approach was extended to MTAP and MTANs¹³⁻¹⁶. Derivatized ImmucillinA (ImmA) and DADMe-ImmucillinA (DADMe-ImmA) provide two generations of transition state analogues developed for MTAP and MTANs (FIG. 2)^{21,22}. ImmA derivatives mimic transition states with partial bond order between the ribosyl group and the adenine while DADMe-ImmA derivatives resemble transition states with a fully dissociated adenine leaving group from the ribosyl cation. In late, dissociative MTAN transition states, C1′ of the ribosyl group is cationic, which resembles the cationic N1′ of DADMe-ImmA. The methylene group between 9-deazaadenine and the pyrrolidine ring in DADMe-ImmA provides geometric similarity between the adenine leaving group and the ribooxacarbenium site, and the 9-deazaadenine provides chemical stability and mimics the increased pKa at N7 found at the MTAN transition states.

Several ImmA and DADMe-ImmA derivatives have been synthesized and tested against MTAP and MTANs, exhibiting some of the highest affinities ever achieved for noncovalent enzyme-inhibitor interactions²³⁻²⁶. For instance, para-chloro-phenylthio-DADMe-ImmA inhibits purified EcMTAN with a dissociation constant of 47 fM, approaching a Kₘ/Kᵢ value of ˜10^{8 23}. Methylthio-DADMe-ImmA inhibits purified human MTAP with 86 pM affinity, and induces apoptosis in cultured head and neck squamous cell carcinoma cell lines without affecting normal human fibroblast cell lines and suppresses tumor growth in mouse xenografts¹².

With the growing global threat of multi-drug resistance, nonconventional antibacterial discovery approaches are required that are nonlethal to bacteria where the potential to develop drug resistance is assumed to be less significant. The present invention satisfies that need.

### SUMMARY OF THE INVENTION

The invention provides methods for treating bacterial infections in a subject comprising administering to the subject a sub-growth inhibiting amount of a 5′-Methylthioadenosine/S-adenosyl homocysteine nucleosidase (MTAN) inhibitor. The invention also provides pharmaceutical compositions comprising a sub-bacterial-growth inhibiting amount of a 5′-Methylthioadenosine/S-adenosyl homocysteine nucleosidase (MTAN) inhibitor and a pharmaceutically acceptable carrier.

### BRIEF DESCRIPTION OF THE FIGURES

FIG. 1. Role of MTAN in bacterial utilization of SAM. This scheme shows the pathways connecting DNA methylation (A), polyamine synthesis (B), autoinducer production (C), and methionine and adenine salvage. A synthase catalyzes the transfer of the amino acid moiety of SAM to an acyl acceptor to produce homoserine lactones in the synthesis of AI-1 molecules, and MTA as by-product. In methyltransferase reactions, SAM produces SAH which is a precursor in the tetrahydrofuran synthesis of AI-2 molecules (shown here as furanosyl boron diester). AI-1 and AI-2 are autoinducers used in bacterial quorum sensing, and MTAN offers a means to block formation of these signaling molecules.

FIG. 2. The reaction catalyzed by MTAN with MTA as substrate, showing a dissociative transition state for E. coli with ribooxacarbenium ion character (top). Structures of stable analogues for an early transition state (ImmucillinA), and a late transition state (DADMe-ImmucillinA) depict differences in bond distances between the adenine leaving group and the ribosyl group, as well as charge localization (bottom). Derived from reference¹³.

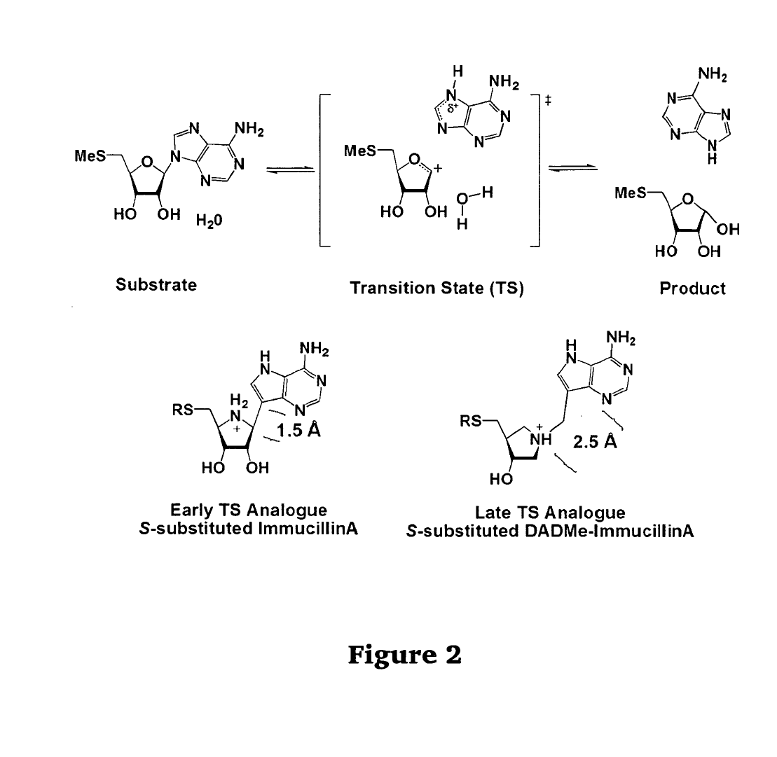

FIGS. 3a-3d. Activity profiles as a function of inhibitor concentration all show dose-dependent drops. (a) Purified VcMTAN inhibition assay with MT-DADMe-ImmA gives an overall dissociation constant Kᵢ* of 73 pM. (b) MTAN activity assay in V. cholerae N16961 cells with BuT-DADMe-ImmA reveals inhibition of adenine production and IC₅₀ fit gives a value of 6 nM. Total ¹⁴C counts did not vary significantly showing efficient recovery of radiolabel. (c) Autoinducer production with EtT-DADMe-ImmA using luminescence induction assay in V. harveyi BB120 resulted in an IC₅₀ of 14 nM. (d) OD₆₀₀ profile of V. cholerae N 16961 grown in the presence of MT-DADMe-ImmA, EtT-DADMe-ImmA, and BuT-DADMe-ImmA (left, middle and right columns, respectively), demonstrate nontoxicity despite remarkable inhibition in cellular MTAN activity and autoinducer production. Complete set of inhibition constants presented in Table 2.

FIGS. 4a-4d. Crystal structure of VcMTAN in complex with BuT-DADMe-ImmA. (a) Overall structure of the VcMTAN structure showing the asymmetric unit content with the inhibitor BuT-DADMe-ImmA bound in the active sites. (b) The active site of the VcMTAN with a 2Fo-Fc map contoured at 1.2σ surrounding the BuT-DADMe-ImmA inhibitor and the proposed nucleophilic water molecule. (c) Space filling picture of the active site of VcMTAN with BuT-DADMe-ImmA in the active site. Hydrophobic parts of the protein (on left side) interact with hydrophobic parts of the inhibitor. Parts of the protein that contain charged residues (dark stick figures) interact with polar groups of the inhibitor. The central mesh-like structure represents loop regions. (d) Schematic drawing of the BuT-DADMe-ImmA inhibitor bound in the active site of VcMTAN.

FIGS. 5a-5b. Comparisons between EcMTAN and VcMTAN structures. (a) Active site superposition of VcMTAN bound to BuT-DADMe-ImmA compared to the active site of EcMTAN bound to MT-DADMe-ImmA. (b) Overall structure of VcMTAN in complex with BuT-DADMe-ImmA with mapped amino acid differences compared to EcMTAN.

FIG. 6. Autoinducer-2 production in wild-type E. coli, wild-type with 0.5 μM BuT-DADMe-ImmA, and an MTAN knockout strain using luminescence induction in V. harveyi BB170. Defective AI-2 production is seen in both the MTAN knockout mutant and the wild-type inhibited with the transition state analogue. Growth phenotypes were nearly identical in the three samples (inset).

### DETAILED DESCRIPTION OF THE INVENTION

The invention provides a method for treating a bacterial infection in a subject comprising administering to the subject a sub-growth inhibiting amount of a 5′-Methylthioadenosine/S-adenosyl homocysteine nucleosidase (MTAN) inhibitor.

As used herein, to treat a bacterial infection in a subject means to reduce the virulence of the bacteria in the subject. The term “bacterial infection” shall mean any deleterious presence of bacteria in a subject. Examples of bacteria capable of causing infections include, but are not limited to Streptococcus pneumoniae, Neisseria meningitides, Pseudomonas aeruginosa, Klebsiella pneumoniae, Staphylococcus aureus, Helicobacter pylori and Escherichia coli.

The term “sub-growth inhibiting amount” of a MTAN inhibitor as used herein means an amount of the inhibitor, which when contacted with a population of bacteria, does not reduce the growth of the bacterial population. Preferably, the sub-growth inhibiting amount of the MTAN inhibitor inhibits quorum sensing in the bacteria. Preferably, the sub-growth inhibiting amount of the MTAN inhibitor is effective to reduce virulence of the bacteria without promoting the development of resistance by the bacteria to the MTAN inhibitor.

The term “quorum sensing” as used herein refers to the process by which bacteria produce and detect signaling molecules with which to coordinate gene expression and regulate processes beneficial to the microbial community. The term “inhibit quorum sensing” as used herein means altering this process such that coordination of gene expression and process regulation in microbial communities are impaired or prevented.

The invention also provides a pharmaceutical composition comprising a sub-bacterial-growth inhibiting amount of a 5′-Methylthioadenosine/S-adenosyl homocysteine nucleosidase (MTAN) inhibitor and a pharmaceutically acceptable carrier. Preferably, the pharmaceutical composition is formulated in dosage form.

As used herein, “pharmaceutically acceptable carriers” are materials that (i) are compatible with the other ingredients of the composition without rendering the composition unsuitable for its intended purpose, and (ii) are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include any of the standard pharmaceutical carriers such as phosphate buffered saline solutions, water, and emulsions such as oil/water emulsions and microemulsions.

Many MTAN inhibitors are known in the art and can be utilized in the methods and compositions of the present invention. Preferred MTAN inhibitors include, but are not limited to, 5′-methylthio-(MT-) DADMe-ImmucillinA, 5′-ethylthio-(EtT-) DADMe-ImmucillinA and 5′-butylthio-(BuT-)DADMe-ImmucillinA. Additional MTAN inhibitors are described below. MTAN inhibitors are described, for example, in U.S. Patent Application Publication No. 2006/0160765 A1; PCT International Patent Application Publication Nos. WO 2006/123953 A1, WO 2007/069923 A1, WO 2007/097648 A1, WO 2008/030118 and WO 2008/079028; and U.S. Pat. Nos. 5,985,848, 7,098,334, and 7,109,331, the contents of which are herein incorporated by reference.

### DEFINITIONS as Applied to MTAN Inhibitors:

The term “alkyl” is intended to include straight- and branched-chain alkyl groups, as well as cycloalkyl groups. The same terminology applies to the non-aromatic moiety of an aralkyl radical. Examples of alkyl groups include, but are not limited to: methyl group, ethyl group, n-propyl group, iso-propyl group, n-butyl group, iso-butyl group, sec-butyl group, t-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group and 1-methyl-2-ethylpropyl group.

The term “alkenyl” means any hydrocarbon radical having at least one double bond, and having up to 30 carbon atoms, and includes any C₂-C₂₅, C₂-C₂₀, C₂-C₁₅, C₂-C₁₀, or C₂-C₆ alkenyl group, and is intended to include both straight- and branched-chain alkenyl groups. The same terminology applies to the non-aromatic moiety of an aralkenyl radical. Examples of alkenyl groups include but are not limited to: ethenyl group, n-propenyl group, iso-propenyl group, n-butenyl group, iso-butenyl group, sec-butenyl group, t-butenyl group, n-pentenyl group, 1,1-dimethylpropenyl group, 1,2-dimethylpropenyl group, 2,2-dimethylpropenyl group, 1-ethylpropenyl group, 2-ethylpropenyl group, n-hexenyl group and 1-methyl-2-ethylpropenyl group.

The term “alkynyl” means any hydrocarbon radical having at least one triple bond, and having up to 30 carbon atoms, and includes any C₂-C₂₅, C₂-C₂₀, C₂-C₁₅, C₂-C₁₀, or C₂-C₆ alkynyl group, and is intended to include both straight- and branched-chain alkynyl groups. The same terminology applies to the non-aromatic moiety of an aralkynyl radical. Examples of alkynyl groups include but are not limited to: ethynyl group, n-propynyl group, iso-propynyl group, n-butynyl group, iso-butynyl group, sec-butynyl group, t-butynyl group, n-pentynyl group, 1,1-dimethylpropynyl group, 1,2-dimethylpropynyl group, 2,2-dimethylpropynyl group, 1-ethylpropynyl group, 2-ethylpropynyl group, n-hexynyl group and 1-methyl-2-ethylpropynyl group.

The term “aryl” means an aromatic radical having 6 to 18 carbon atoms and includes heteroaromatic radicals. Examples include monocyclic groups, as well as fused groups such as bicyclic groups and tricyclic groups. Examples include but are not limited to: phenyl group, indenyl group, 1-naphthyl group, 2-naphthyl group, azulenyl group, heptalenyl group, biphenyl group, indacenyl group, acenaphthyl group, fluorenyl group, phenalenyl group, phenanthrenyl group, anthracenyl group, cyclopentacyclooctenyl group, and benzocyclooctenyl group, pyridyl group, pyrrolyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, furyl group, pyranyl group, benzofuryl group, isobenzofuryl group, thienyl group, thiazolyl group, isothiazolyl group, benzothiazolyl group, oxazolyl group, and isoxazolyl group.

The term “aralkyl” means an alkyl radical having an aryl substituent.

The term “alkoxy” means an hydroxy group with the hydrogen replaced by an alkyl group.

The term “halogen” includes fluorine, chlorine, bromine and iodine.

The term “prodrug” as used herein means a pharmacologically acceptable derivative of the MTAN inhibitor, such that an in vivo biotransformation of the derivative gives the MTAN inhibitor. Prodrugs of MTAN inhibitors may be prepared by modifying functional groups present in the compounds in such a way that the modifications are cleaved in vivo to give the parent compound.

In one embodiment, as described in U.S. Patent Application Publication No. 2006/0160765 A1 and in PCT International Patent Application Publication No. WO 2007/097648 A1, the MTAN inhibitor comprises a compound having formula (I):

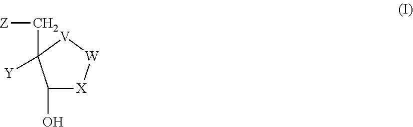

wherein V is selected from CH₂ and NH, and W is selected from NR¹ and NR²; or V is selected from NR¹ and NR², and W is selected from CH₂ and NH; X is selected from CH₂ and CHOH in the R or S-configuration; Y is selected from hydrogen, halogen and hydroxy, except where V is selected from NH, NR¹ and NR² then Y is hydrogen; Z is selected from hydrogen, halogen, hydroxy, SQ, OQ and Q, where Q is an optionally substituted alkyl, aralkyl or aryl group, each of which is optionally substituted with one or more substituents selected from hydroxy, halogen, methoxy, amino, or carboxy; R¹ is a radical of the formula (II)

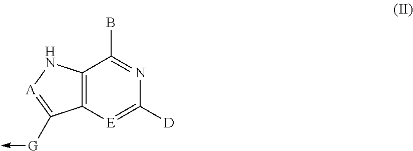

R² is a radical of the formula (III)

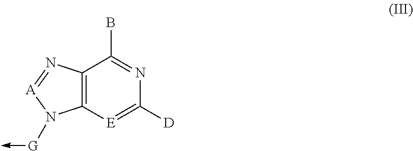

A is selected from N, CH and CR, where R is selected from halogen, optionally substituted alkyl, aralkyl or aryl, each of which is optionally substituted with one or more substituents selected from hydroxy and halogen, OH, NH₂, NHR³, NR³R⁴ and SR⁵, where R³, R⁴ and R⁵ are each optionally substituted alkyl, aralkyl or aryl groups, each of which is optionally substituted with one or more substituents selected from hydroxy and halogen; B is selected from OH, NH₂, NHR⁶, SH, hydrogen and halogen, where R⁶ is an optionally substituted alkyl, aralkyl or aryl group, each of which is optionally substituted with one or more substituents selected from hydroxy and halogen; D is selected from OH, NH₂, NHR⁷, hydrogen, halogen and SCH₃, where R⁷ is an optionally substituted alkyl, aralkyl or aryl group, each of which is optionally substituted with one or more substituents selected from hydroxy and halogen; E is selected from N and CH; G is selected from CH₂ and NH, or G is absent, provided that where W is NR¹ or NR² and G is NH then V is CH₂, and provided that where V is NR¹ or NR² and G is NH then W is CH₂; or a tautomer thereof, or a pharmaceutically acceptable salt thereof, or an ester thereof, or a prodrug thereof.

Preferably, Z is selected from hydrogen, halogen, hydroxy, SQ and OQ. More preferably, Z is OH. Alternatively it is preferred that Z is SQ. In another preferred embodiment, Z is Q.

It is also preferred that V is CH₂. It is further preferred that X is CH₂. Additionally, it is preferred that G is CH₂.

It is also preferred that where W is selected from NH, NR¹ or NR², then X is CH₂.

Preferred compounds of the invention include those where V, X and G are all CH₂, Z is OH and W is NR¹. Other preferred compounds of the invention include those where V, X and G are all CH₂, Z is SQ and W is NR¹.

Preferably Y is hydrogen. Alternatively, it is preferred that Y is hydroxy.

Preferably B is hydroxy. Alternatively, it is preferred that B is NH₂.

Preferably A is CH. Alternatively, it is preferred that A is N.

Preferably D is H. Alternatively, it is preferred that D is NH₂.

It is also preferred that E is N.

Preferably, Q is alkyl, preferably a C₁-C₆ alkyl group such as methyl, ethyl or butyl.

Preferably, the aryl group is a phenyl or benzyl group.

Preferred compounds include those having the formula:

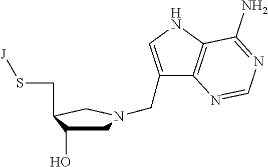

where J is aryl, aralkyl or alkyl, each of which is optionally substituted with one or more substituents selected from hydroxy, halogen, methoxy, amino, and carboxy; or a pharmaceutically acceptable salt thereof, or a prodrug thereof.

Preferred compounds include those where J is C₁-C₇ alkyl, such as, for example, J is methyl, ethyl, n-propyl, i-propyl, n-butyl, cyclobutyl, cyclopentyl, cyclohexyl, cyclohexylmethyl, or cycloheptyl. Other preferred compounds include those where J is phenyl, optionally substituted with one or more halogen substituents, such as, for example, J is phenyl, p-chlorophenyl, p-fluorophenyl, or m-chlorophenyl. Other preferred compounds include those where J is heteroaryl, 4-pyridyl, aralkyl, benzylthio, or —CH₂CH₂(NH₂)COOH.

Examples of MTAN inhibitors include, but are not limited to (3R,4S)-1-[(9-deazaadenin-9-yl)methyl]-3-hydroxy-4-(methylthiomethyl)pyrrolidine; (3R,4S)-1-[(9-deazaadenin-9-yl)methyl]-3-hydroxy-4-(benzylthiomethyl)pyrrolidine; (3R,4S)-1-[(8-Aza-deazaadenin-9-yl)methyl]-3-hydroxy-4-(benzylthiomethyl)pyrrolidine hydrochloride; (3R,4S)-1-[(9-deazaadenin-9-yl)methyl]-3-hydroxy-4-(4-chlorophenylthiomethyl)pyrrolidine; and (3R,4S)-1-[(9-deazaadenin-9-yl)methyl]-3-hydroxy-4-(2-phenylethyl)pyrrolidine hydrochloride.

In another embodiment, as described in PCT International Patent Application Publication No. WO 2007/069923 A1, the MTAN inhibitor comprises a compound having formula (IV):

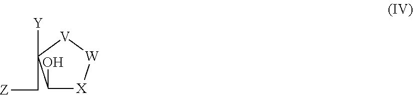

wherein V is selected from CH₂ and NH, and W is selected from NR¹ and NR²; or V is selected from NR¹ and NR², and W is selected from CH₂ and NH; X is selected from CH₂ and CHOH in the R or S-configuration; Y is selected from hydrogen, halogen and hydroxy, except where V is selected from NH, NR¹ and NR² then Y is hydrogen; Z is selected from hydrogen, halogen, hydroxy, SQ, OQ and Q, where Q is an optionally substituted alkyl, aralkyl or aryl group; R¹ is a radical of the formula (V)

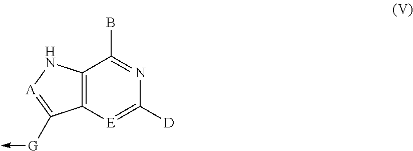

R² is a radical of the formula (VI)

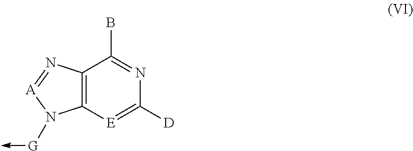

A is selected from N, CH and CR, where R is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups; B is selected from OH, NH₂, NHR⁶, SH, hydrogen and halogen, where R⁶ is an optionally substituted alkyl, aralkyl or aryl group; D is selected from OH, NH₂, NHR⁷, hydrogen, halogen and SCH₃, where R⁷ is an optionally substituted alkyl, aralkyl or aryl group; E is selected from N and CH; G is selected from CH₂ and NH, or G is absent, provided that where W is NR¹ or NR² and G is NH then V is CH₂, and provided that where V is NR¹ or NR² and G is NH then W is CH₂; or a tautomer thereof, or a pharmaceutically acceptable salt thereof, or an ester thereof, or a prodrug thereof.

Preferably, Z is selected from hydrogen, halogen, hydroxy, SQ and OQ. More preferably, Z is OH. Alternatively it is preferred that Z is SQ. In another preferred embodiment, Z is Q.

It is also preferred that V is CH₂. It is further preferred that X is CH₂. Additionally, it is preferred that G is CH₂.

Preferably W is NR¹. Alternatively it is preferred that W is NR². It is also preferred that where W is selected from NH, NR¹ or NR², then X is CH₂.

Preferred compounds of the invention include those where V, X and G are all CH₂, Z is OH and W is NR¹.

Other preferred compounds of the invention include those where V, X and G are all CH₂, Z is SQ and W is NR¹.

Preferably Y is hydrogen. Alternatively, it is preferred that Y is hydroxy.

Preferably B is hydroxy. Alternatively, it is preferred that B is NH₂.

Preferably A is CH. Alternatively, it is preferred that A is N.

Preferably D is H. Alternatively, it is preferred that D is NH₂.

It is also preferred that E is N.

It is preferred that any halogen is selected from chlorine and fluorine.

Q may be substituted with one or more substituents selected from OH, halogen (particularly fluorine or chlorine), methoxy, amino or carboxy.

R3, R4, R5, R6 and R7 may each be substituted with one or more substituents selected from OH or halogen, especially fluorine or chlorine.

In another embodiment, as described in PCT International Patent Application Publication No. WO 2006/123953 A1, the MTAN inhibitor comprises a compound having formula (VII):

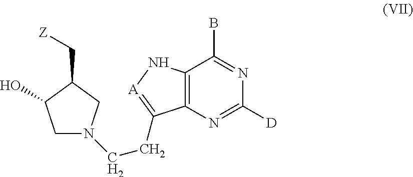

wherein:
A is N or CH; B is OH or NH₂; D is H, OH, NH₂ or SCH₃; and Z is OH or SQ, where Q is an optionally substituted alkyl, aralkyl, or aryl group; or a tautomer thereof; or a pharmaceutically acceptable salt thereof; or an ester prodrug form thereof.

Preferred compounds include those where Z is OH, A is CH, B is OH, and D is H or NH₂. Other preferred compounds include those where Z is SQ, A is CH, B is NH₂, and D is H.

In another embodiment, as described in U.S. Pat. No. 7,098,334, the MTAN inhibitor comprises a compound having formula (VIII):

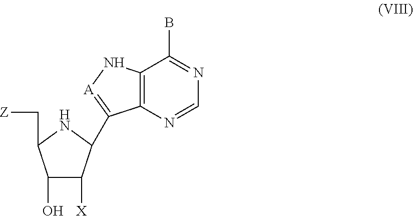

wherein:
A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups; B is selected from NH₂ and NHR⁴, where R⁴ is an optionally substituted alkyl, aralkyl or aryl group; X is selected from H, OH and halogen; and Z is selected from H, Q, SQ and OQ, where Q is an optionally substituted alkyl, aralkyl or aryl group; or a tautomer thereof; or a pharmaceutically acceptable salt thereof; or an ester thereof; or a prodrug thereof; with the proviso that the stereochemistry of the aza-sugar moiety is D-ribo or 2′-deoxy-D-erythro-.

Preferably, A is CH. More preferably Z is SQ when A is CH.

It is also preferred that B is NH₂. More preferably Z is SQ when B is NH₂. Still more preferably Q is C₁-C₅ alkyl or C₂-C₅ alky when B is NH₂ and Z is SQ.

It is further preferred that A is N. More preferably Z is SQ when A is N. Still more preferably Q is C₁-C₅ alkyl or C₂-C₅ alky when A is N and Z is SQ.

Preferably X is OH.

It is also preferred that Z is SQ. More preferably Q is C₁-C₅ alkyl when Z is SQ. Still more preferably Q is an optionally substituted aryl group when Z is SQ.

Preferred compounds include those where Q is selected from phenyl, 3-chlorophenyl, 4-chlorophenyl, 4-fluorophenyl, 3-methylphenyl, 4-methylphenyl, benzyl, hydroxyethyl, fluoroethyl, naphthyl, methyl and ethyl.

Further examples of MTAN inhibitors include 5′-phenylthio-ImmucillinA; 5′-methylthio-ImmucillinA; 5′-ethylthio-ImmucillinA; 5′-deoxy-5′-ethyl-ImmucillinA; 5′-methylthio-8-aza-ImmucilinA; 5′-hydroxyethylthio-ImmucillinA; 5′fluoroethylthio-ImmucillinA; 5′-deoxy-ImmucilinA; 5′-methoxy-ImmucillinA; 5′-(p-fluorophenyl-thio-ImmucillinA; 5′-(p-chlorophenyl-thio)-ImmucillinA; 5′-(m-chlorophenyl-thio)-ImmucillinA; 5′-benzylthio-ImmucillinA; 5′-(m-tolylthio)-ImmucillinA; 5′-(p-tolylthio)-ImmucillinA; and 5′-napthylthio-ImmucillinA.

In another embodiment, as described in U.S. Pat. No. 7,109,331, the MTAN inhibitor comprises a compound having formula (IX):

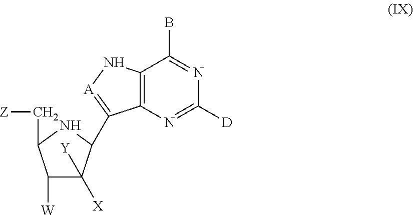

wherein:
A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups; B is selected from OH, NH₂, NHR⁴, H and halogen, where R⁴ is an optionally substituted alkyl, aralkyl or aryl group; D is selected from OH, NH₂, NHR⁵, H, halogen and SCH₃, where R⁵ is an optionally substituted alkyl, aralkyl or aryl group; X and Y are independently selected from H, OH and halogen, with the proviso that when one of X and Y is hydroxy or halogen, the other is hydrogen; Z is OH, or, when X is hydroxy, Z is selected from hydrogen, halogen, hydroxy, SQ and OQ, where Q is an optionally substituted alkyl, aralkyl or aryl group; and W is OH or H, with the proviso that when W is OH, then A is CR where R is as defined above; or a tautomer thereof; or a pharmaceutically acceptable salt thereof; or an ester thereof; or a prodrug thereof.

Preferably B is OH. Preferably when B is NHR⁴ and/or D is NHR⁵, then R⁴ and/or R⁵ are C₁-C₄ alkyl.

Preferably when one or more halogens are present they are chosen from chlorine and fluorine.

Preferably when Z is SQ or OQ, Q is C₁-C₅ alkyl or phenyl.

Preferably D is H, or when D is other than H, B is OH.

More preferably, B is OH, D is H, OH or NH₂, X is OH or H, Y is H, most preferably with Z as OH, H, or methylthio, especially OH.

In certain preferred embodiments W is OH, Y is H, X is OH, and A is CR where R is methyl or halogen, preferably fluorine.

In other preferred embodiments, W is H, Y is H, X is OH and A is CH.

In another embodiment, as described in U.S. Pat. No. 5,985,848 and in PCT International Patent Application Publication No. WO 2007/097648 A1, the MTAN inhibitor comprises a compound having formula (X):

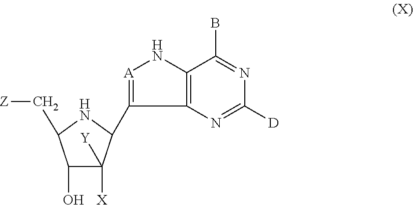

wherein A is CH or N; B is chosen from OH, NH₂, NHR, H or halogen; D is chosen from OH, NH₂, NHR, H, halogen or SCH₃; R is an optionally substituted alkyl, aralkyl or aryl group; and X and Y are independently selected from H, OH or halogen except that when one of X and Y is hydroxy or halogen, the other is hydrogen; and Z is OH or, when X is hydroxy, Z is selected from hydrogen, halogen, hydroxy, SQ or OQ where Q is an optionally substituted alkyl, aralkyl or aryl group; or a tautomer thereof; or a pharmaceutically acceptable salt thereof; or an ester thereof; or a prodrug thereof.

Preferably when either of B and/or D is NHR, then R is C₁-C₄ alkyl.

Preferably when one or more halogens are present they are chosen from chlorine and fluorine.

Preferably when Z is SQ or OQ, Q is C₁-C₅ alkyl or phenyl.

Preferably D is H, or when D is other than H, B is OH.

More preferably, B is OH, D is H, OH or NH₂, X is OH or H, Y is H, most preferably with Z as OH, H or methylthio, especially OH.

Preferred compounds include those having the formula:

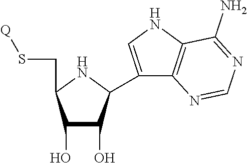

where Q is aryl, aralkyl or alkyl, each of which is optionally substituted with one or more substituents selected from hydroxy, halogen, methoxy, amino, carboxy, and straight- or branched-chain C₁-C₆ alkyl; or a pharmaceutically acceptable salt thereof, or a prodrug thereof. Preferred compounds include those where Q is methyl, ethyl, 2-fluoroethyl, or 2-hydroxyethyl; phenyl, naphthyl, p-tolyl, m-tolyl, p-chlorophenyl, m-chlorophenyl or p-fluorophenyl; or aralkyl such as, for example, benzyl.

In another embodiment, as described in PCT International Patent Application Publication No. WO 2008/030118, the MTAN inhibitor comprises a compound having formula (XI):

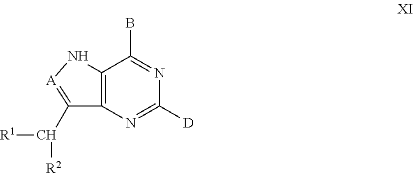

wherein R¹ is H or NR³R⁴; R² is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, amino, hydroxy, or alkoxy groups; provided that when R¹ is H, R² is an alkyl, alkenyl, alkynyl, aralkyl, aralkenyl, aralkynyl, or aryl group which is substituted with at least one NR³R⁴ group; R³ and R⁴, independently of each other, is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, hydroxy, or alkoxy groups; A is N or CH; B is NH₂ or NHR⁵, R⁵ is an alkyl, alkenyl, alkynyl, aralkyl, aralkenyl, aralkynyl, or aryl group, each of which is optionally substituted with one or more halogen or hydroxy groups; and D is H, OH, NH₂, or SCH₃; or a tautomer thereof, or a pharmaceutically acceptable salt thereof, or an ester prodrug form thereof.

When R¹ is H, then R² is preferably alkyl substituted with at least one NR³R⁴ group.

When R³ or R⁴ is optionally substituted alkyl, the alkyl group is preferably substituted by one or more hydroxy groups. For example, R³ or R⁴ may be hydroxymethyl, hydroxyethyl, hydroxypropyl, dihydroxypropyl, hydroxybutyl, dihydroxybutyl, trihydroxybutyl, hydroxypentyl, dihydroxypentyl, or trihydroxpentyl.

R³ or R⁴ may also preferably be alkyl substituted by one or more hydroxy groups and/or one or more optionally substituted thiol, alkylthio, arylthio, or aralkylthio groups. For example, R³ or R⁴ may be methylthiomethyl, methylthioethyl, methylthiopropyl, methylthiohydroxypropyl, methylthiodihydroxypropyl, methylthiobutyl, methylthiohydroxybutyl, methylthiodihydroxybutyl, methylthiotrihydroxybutyl, methylthiopentyl, methylthiohydroxypentyl, methylthiodihydroxypentyl, methylthiotrihydroxypentyl or methylthiotetrahydroxypentyl.

When R¹ is NR³R⁴, and R³ and R⁴ are H, R² is preferably an optionally substituted alkyl, more preferably an optionally substituted C₁-C₅ alkyl, for example, hydroxymethyl, hydroxyethyl, hydroxypropyl, dihydroxypropyl, hydroxybutyl, dihydroxybutyl, trihydroxybutyl, hydroxypentyl, dihydroxypentyl, trihydroxpentyl, methylthiomethyl, methylthioethyl, methylthiopropyl, methylthiohydroxypropyl, methylthiodihydroxypropyl, methylthiobutyl, methylthiohydroxybutyl, methylthiodihydroxybutyl, methylthiotrihydroxybutyl, methylthiopentyl, methylthiohydroxypentyl, methylthiodihydroxypentyl, methylthiotrihydroxypentyl or methylthiotetrahydroxypentyl.

When R¹ is NR³R⁴, and R³ is H and R⁴ is an optionally substituted alkyl, R² is preferably H.

When R¹ is NR³R⁴, and R³ is H and R⁴ is an optionally substituted alkyl, R² is preferably an optionally substituted alkyl, more preferably an optionally substituted C₁-C₅ alkyl.

When R¹ is NR³R⁴, and R³ and R⁴ are each an optionally substituted alkyl, R² is preferably H.

Preferrably, B is NH₂.

It is further preferred that D is H. Alternatively, D may preferably be OH, NH₂ or SCH₃.

Further examples of MTAN inhibitors include 2-((4-amino-5H-pyrrolo[3,2-d]pyrimidin-7-yl)methylamino)-2-(methylthiomethyl)propane-1,3-diol; (S)-1-((4-amino-5H-pyrrolo[3,2-d]pyrimidin-7-yl)methylamino)-3-(methylthio)propan-2-ol; (2RS,3SR)-4-[(4-amino-5H-pyrrolo[3,2-d]pyrimidin-7-yl)methylamino)-3-(methylthiomethyl)butane-1,2-diol; (2R,3S)-4-(((4-amino-5H-pyrrolo[3,2-d]pyrimidin-7-yl)methyl)(methyl)amino)-3-(methylthiomethyl)butane-1,2-diol; (2R,3R)-2-((4-amino-5H-pyrrolo[3,2-d]pyrimidin-7-yl)methylamino)-4-(methylthio)butane-1,3-diol; (2R,3S)-2-((4-amino-5H-pyrrolo[3,2-d]pyrimidin-7-yl)methylamino)-4-(methylthio)butane-1,3-diol; (2S,3R)-2-((4-amino-5H-pyrrolo[3,2-d]pyrimidin-7-yl)methylamino)-4-(methylthio)butane-1,3-diol; (2R,3S)-2-(((4-amino-5H-pyrrolo[3,2-d]pyrimidin-7-yl)methyl)(methyl)amino)-4-(methylthio)butane-1,3-diol; (2RS,3RS)-2-{[(4-amino-5H-pyrrolo[3,2-d]-pyrimidin-7-yl)methylamino]methyl}-4-(methylthio)butane-1,3-diol; (2RS,3RS)-2-((((4-amino-5H-pyrrolo[3,2-d]pyrimidin-7-yl)methyl)(methyl)amino)methyl)-4-(methylthio)butane-1,3-diol; (2S,3R)-1-(((4-amino-5H-pyrrolo[3,2-d]pyrimidin-7-yl)methyl)(methyl)amino)-4-(methylthio)butane-2,3-diol; and (S)-2-((S)-1-(4-amino-5H-pyrrolo[3,2-d]pyrimidin-7-yl)-2-hydroxyethylamino)-3-(methylthio)propan-1-ol.

In another embodiment, as described in PCT International Patent Application Publication No. WO 2008/079028, the MTAN inhibitor comprises a compound having formula (XII):

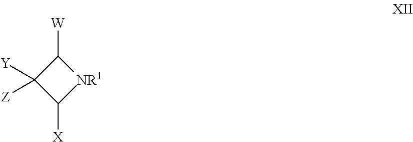

wherein W and X are each independently selected from hydrogen, CH₂OH, CH₂OQ and CH₂SQ; Y and Z are each independently selected from hydrogen, halogen, CH₂OH, CH₂OQ, CH₂SQ, SQ, OQ and Q; Q is an alkyl, aralkyl or awl group each of which may be optionally substituted with one or more substituents selected from hydroxy, halogen, methoxy, amino, or carboxy; R¹ is a radical of the formula (XIII)

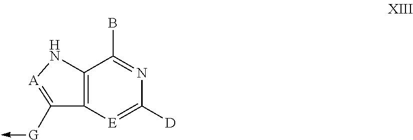

or R¹ is a radical of the formula (XIV)

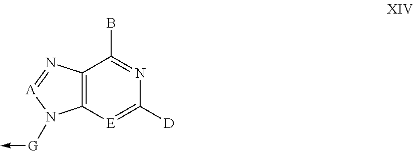

A is selected from N, CH and CR², where R² is selected from halogen, alkyl, aralkyl, aryl, OH, NH₂, NHR³, NR³R⁴ and SR⁵, where R³, R⁴ and R⁵ are each alkyl, aralkyl or aryl groups optionally substituted with hydroxy or halogen, and where R² is optionally substituted with hydroxy or halogen when R² is alkyl, aralkyl or aryl; B is selected from hydroxy, NH₂, NHR⁶, SH, hydrogen and halogen, where R⁶ is an alkyl, aralkyl or aryl group optionally substituted with hydroxy or halogen; D is selected from hydroxy, NH₂, NHR⁷, hydrogen, halogen and SCH₃, where R⁷ is an alkyl, aralkyl or aryl group optionally substituted with hydroxy or halogen; E is selected from N and CH; G is a C₁₋₄ saturated or unsaturated alkyl group optionally substituted with hydroxy or halogen, or G is absent; or a tautomer thereof, or a pharmaceutically acceptable salt thereof, or an ester thereof, or a prodrug thereof.

Preferably Z is selected from hydrogen, halogen, CH₂OH, CH₂OQ and CH₂SQ. More preferably Z is CH₂OH. Alternatively it is preferred that Z is CH₂SQ. In another preferred embodiment, Z is Q.

It is preferred that G is CH₂.

R¹ may be a radical of the formula (XIII) or, alternatively, may be a radical of formula (XIV).

Preferred compounds include those where one of Y and Z is CH₂OQ and the other is hydrogen. Other preferred compounds include those where one of Y and Z is CH₂SQ and the other is hydrogen.

B is preferably hydroxy or NH₂. A is preferably CH or N. D is preferably H or NH₂. It is also preferred that E is N.

It is preferred that when any of Y, Z, B and D is halogen, each halogen is independently chlorine or fluorine.

Examples of MTAN inhibitors include 1-[9-deazaadenin-9-yl)methyl]-3-methylthiomethylazetidine-3-methanol hydrochloride and 1-[9-deazaadenin-9-yl)methyl]-3-methylthiomethylazetidine.

Another example of an MTAN inhibitor is 2-amino-4-[5-(4-amino-5H-pyrrolo[3,2-d]pyrimidin-7-yl)-3,4-dihydroxypyrrolidin-2-ylmethylsulfanyl]-butyric acid⁴⁶.

The active compounds may be administered to a patient by a variety of routes, including orally, parenterally, by inhalation spray, topically, rectally, nasally, buccally or via an implanted reservoir. The specific dosage required for any particular patient will depend upon a variety of factors, including the patient's age and body weight.

For oral administration the compounds can be formulated into solid or liquid preparations, for example tablets, capsules, powders, solutions, suspensions and dispersions. Such preparations are well known in the art as are other oral dosage regimes not listed here. In the tablet form the compounds may be tableted with conventional tablet bases such as lactose, sucrose and corn starch, together with a binder, a disintegration agent and a lubricant. The binder may be, for example, corn starch or gelatin, the disintegrating agent may be potato starch or alginic acid, and the lubricant may be magnesium stearate. For oral administration in the form of capsules, diluents such as lactose and dried cornstarch may be employed. Other components such as colourings, sweeteners or flavourings may be added.

When aqueous suspensions are required for oral use, the active ingredient may be combined with carriers such as water and ethanol, and emulsifying agents, suspending agents and/or surfactants may be used. Colourings, sweeteners or flavourings may also be added.

The compounds may also be administered by injection in a physiologically acceptable diluent such as water or saline. The diluent may comprise one or more other ingredients such as ethanol, propylene glycol, an oil or a pharmaceutically acceptable surfactant.

The compounds may also be administered topically. Carriers for topical administration of the compounds of include mineral oil, liquid petrolatum, white petrolatum, propylene glycol, polyoxyethylene, polyoxypropylene compound, emulsifying wax and water. The compounds may be present as ingredients in lotions or creams, for topical administration to skin or mucous membranes. Such creams may contain the active compounds suspended or dissolved in one or more pharmaceutically acceptable carriers. Suitable carriers include mineral oil, sorbitan monostearate, polysorbate 60, cetyl ester wax, cetearyl alcohol, 2-octyldodecanol, benzyl alcohol and water.

The compounds may further be administered by means of sustained release systems. For example, they may be incorporated into a slowly dissolving tablet or capsule.

The subject to be treated can be an animal or human, and is preferably a human.

The present invention also provides for the use of a subgrowth inhibiting amount of an MTAN inhibitor for treating bacterial infections in a subject. The present invention further provides for the use of a subgrowth inhibiting amount of an MTAN inhibitor for the preparation of a composition for treating bacterial infections in a subject.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

Cell lines, DADMe-ImmucillinAs, xanthine oxidase, radiolabeled MTA. Vibrio cholerae El Tor N16961 was obtained from American Type Culture Collection (Manassas, Va.). Vibrio harveyi BB120 and BB170 were provided by Dr. Michael G. Surette (University of Calgary). Escherichia coli MTAN knockout was provided by Dr. Clive Bradbeer (University of Virginia). DADMe-ImmucillinAs were synthesized as described previously³⁸. Xanthine oxidase was purchased from Sigma (St. Louis, Mo.). [8-¹⁴C]MTA was synthesized as previously described¹².

VcMTAN expression and purification. The gene encoding MTAN in V. cholerae was synthesized and cloned into pDONR221 vector by DNA 2.0 (Menlo Park, Calif.), along with an N-terminal thrombin-cleavable 6-His tag. The gene was placed into pBAD-DEST49 expression vector using Gateway cloning technology (Invitrogen, Carlsbad, Calif.), and was transformed into BL21 Star competent cells. Cells were grown at 37° C. with shaking to OD₆₀₀=0.6, and induction was initiated with 0.05% arabinose and grown for another 4 hours. Harvested cells were lysed using high pressure French press at 15K psi. Cell debris was removed by centrifugation, and the cleared supernatant was loaded on a Ni-Sepharose High Performance His-tag affinity column (GE Healthcare, Piscataway, N.J.). His-tagged MTAN was purified over a gradient of 0-250 mM imidazole, and buffer-exchanged into 100 mM HEPES at pH 7.0 prior to −80° C. storage.

VcMTAN kinetics. Kinetic constants (k_{cat} and Kₘ) for VcMTAN were determined spectrophotometrically by following the loss of MTA at 274 nm (Δε₂₇₄=1.6 mM⁻¹ cm⁻¹). Reactions were carried out at 25° C. in 100 mM HEPES, pH 7.5, and 50 mM KCl with various concentrations of MTA, and initiated by addition of 10 to 12 nM VcMTAN.

Inhibition of purified MTAN activity. Inhibition constants (Kᵢ and Kᵢ*) were determined using a xanthine oxidase-coupled reaction described previously, where adenine produced in the MTAN reaction is converted to 2,8-dihydroxyadenine, monitored at 293 nm²³. Reaction mixtures contained saturating levels of MTA (1 to 2 mM), and various concentrations of methylthio- (MT-), ethylthio- (EtT-), and butylthio- (BuT-) DADMe-ImmA. Samples were prepared in 100 mM HEPES pH 7.5, 50 mM KCl, with ˜0.5 units of xanthine oxidase, and 12 nM VcMTAN to initiate the reaction. Reactions were monitored for 2 hours at 25° C. on a Cary 300 Bio UV-Vis spectrophotometer. Inhibition constants were obtained by fitting the data to the following expression for competitive inhibition using KaleidaGraph 3.6 (Synergy Software, Reading, Pa.):

$${v}_{s}^{′} / {v}_{s} = \frac{{K}_{m} + [ S ]}{{K}_{m} + [ S ] + {K}_{m} [ I ] / {K}_{d}}$$

where νₛ′ and νₛ are steady state rates in the presence, and absence of inhibitor, respectively; Kₘ is the Michaelis constant for substrate MTA which was obtained as described above; [S] and [I] are the concentrations of MTA and inhibitor, respectively. If the concentration of inhibitor is smaller than 10-fold concentration of enzymes, the following correction was then applied:

I′=I−(1−ν₀′/ν₀)Eₜ

where I′ is the effective inhibitor concentration; I is the concentration of inhibitor used in the assay; ν₀′ and νₒ are initial rates in the presence, and absence of inhibitor, respectively; and Eₜ is total MTAN concentration used in the assay.

Crystallization of BuT-DADMe-ImmucillinA—MTAN complex. Purified VcMTAN was concentrated to 15 mg/mL and incubated on ice for 10 minutes with 1 mM BuT-DADMe-ImmA. The VcMTAN-BuT-DADMe-ImmA complex was crystallized using sitting drop vapor diffusion at 18° C. against an 80 μL reservoir containing 0.2 M potassium iodide 20% (w/v) PEG3350, where 1 μL of the protein solution was mixed with 1 μL of the reservoir solution.

Data collection. Crystals were soaked in mother liquor supplemented with 20% glycerol and flash cooled to −178° C. prior to data collection. Diffraction from the VcMTAN-BuT-DADMe-ImmA crystals is consistent with the space group P2₁ (a=67.74, b=54.33, c=69.79 Å and β=115.4) with two molecules in the asymmetric unit. The Matthews coefficient was 2.1 Å³/Da, which corresponds to a solvent content of 41%. Diffraction data were collected to a resolution of 2.3 Å for the complex at beamline X29A at the National Synchrotron Lightsource, Brookhaven National Laboratory using an ADSC Quantum 315 detector. Each frame was exposed for 10 s with an oscillation range of 1°. The HKL2000 suite was used for integration and scaling of the data (Table 1)³⁹.

TABLE 1
Data processing and refinement statistics for
VcMTAN - BuT-DADMe-ImmA complex.

| | |
|---|---|
| Wavelength (Å) | 1.10010 |
| spacegroup | P21 |
| cell: | |
| a) | 67.74 |
| b) | 54.33 |
| c) | 69.79 |
| α) | 90 |
| β) | 115.4 |
| γ) | 90 |
| Resolution (Å) | 50-2.3 (2.83-2.3) |
| Unique reflections | 18733 (1628) |
| Completeness (%)^{a} | 90.8 (79.9) |
| Multiplicity^{a} | 3.8 (2.9) |
| R_{sym} (%)^{a,b} | 6.6 (18.8) |
| I/σ^{a} | 15.1 (4.4) |
| No. of protein atoms | 3410 |
| No. of water | 69 |
| No. of ligands | 2 |
| Add. atoms | 1 Iodine |
| R-factor | 20.0 |
| R-free | 26.4 |
| Average B-factor | 45.95 |
| R.m.s bond (Å) | 0.010 |
| R.m.s angle (Å) | 1.319 |
| Ramachandran analysis | |
| Most favored | 88.9 |
| Allowed | 11.1 |
| Disallowed | 0 |

^{a}Values for the highest resolution shell are given in parentheses.
^{b}R_{sym} = (ΣₕₖₗΣᵢ|Iᵢ(hkl) − <I(hkl)>|)/ΣₕₖₗΣᵢIᵢ(hkl) for n independent reflections and observations of a given reflection, <I(hkl)> is the average intensity of the i observation.

Structure determination and refinement. The structure of the VcMTAN in complex with BuT-DADMe-ImmA was solved by molecular replacement using the MTAN from E. coli (Protein Data Bank ID code 1Z5P.pdb without water) as a search model. Molecular replacement with MOLREP, and refinement with REFMAC5 were carried out using the CCP4i package⁴⁰⁻⁴². COOT was used for molecular modeling⁴³. Clear density was observed in the Fo-Fc maps for the ligands at 3.5σ and they were built into the electron density. The final structure had an R-factor and R-free value of 20%, and 26.4%, respectively. Data processing and refinement statistics are summarized in Table 1. The coordinates and structure factors of VcMTAN in complex with BuT-DADMe-ImmA have been deposited in the protein data bank with accession code 3DP9. All figures were made using PyMOL⁴⁴.

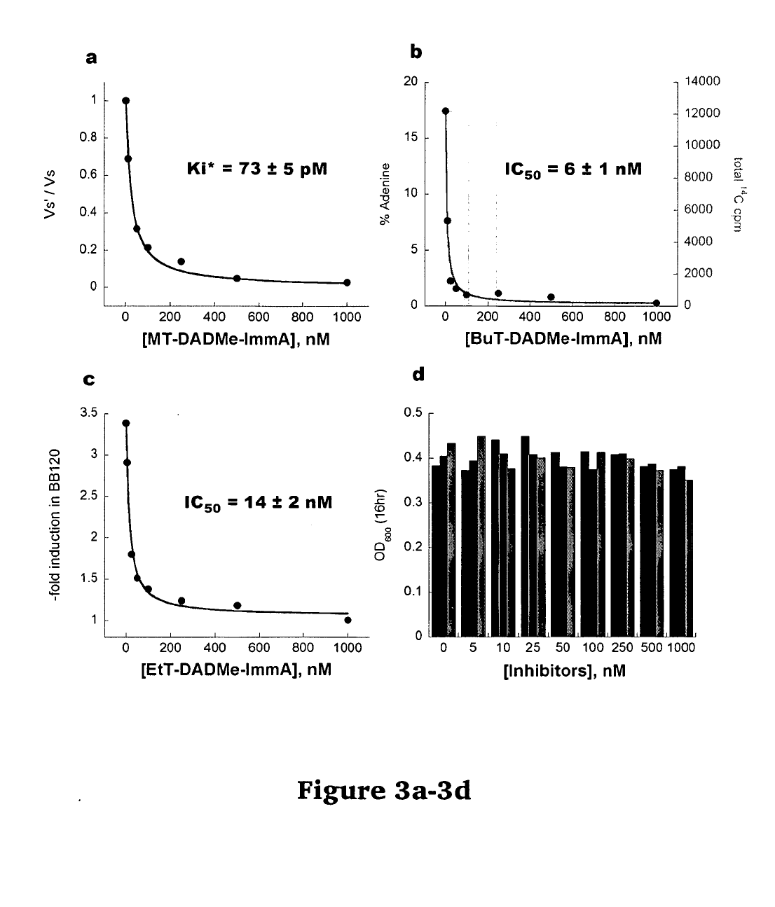

Inhibition of cellular MTAN activity. V. cholerae N16961 cells were grown at 37° C. to stationary phase in LB medium for 16 hours in the absence and presence of 1 to 1000 nM MT-, EtT-, and BuT-DADMe-ImmA. Pelleted cells were washed twice with PBS and lysed with BugBuster Protein Extraction Reagent (Novagen). The lysate was clarified by centrifugation and incubated with [8-¹⁴C]MTA in 50 mM phosphate buffer, pH 7.9, 10 mM KCl at 25° C. for 20 minutes and then quenched with 70% perchloric acid to give a final concentration of 20%. The reaction was neutralized with 45.5% potassium hydroxide, and centrifuged to remove any precipitated salts. Carrier adenine and MTA were added to the cleared supernatant prior to loading on a C₁₈ Luna HPLC column (Phenomenex). ¹⁴C-Adenine product was separated from unreacted MTA using a gradient of 5-60% methanol in 25 mM ammonium acetate, pH 6, and 0.5 mM 1-octanesulfonic acid on a Waters 600 HPLC system with a 2487 Dual λ Absorbance detector set at 261 nm. Adenine eluted first with a retention time of 11 minutes, followed by MTA which eluted at 14 minutes. An additional fraction was collected at around 10 minutes for each run, to determine background counts and ascertain no carryover from the previous run. Fractions were dried using speedvac, and reconstituted in 1 mL deionized water prior to addition of 9 mL Liquiscint scintillation fluid (National Diagnostics). ¹⁴C counts were determined on a Wallac 1414 liquid scintillation counter for 120 minutes per cycle for 2 cycles. Extent of reaction was determined as percentage ¹⁴C-adenine counts of the total combined adenine and unreacted MTA counts. A control run where the cell lysate was replaced with just the lysis buffer prior to addition of radiolabeled substrate was included, and the ¹⁴C counts for spiked cold adenine were used for correction of sample counts. The amount of [8-¹⁴C]MTA used in the assays produced a total of between 11000 to 14000 cpms, showing efficient recovery of radiolabel between adenine and unreacted MTA (FIG. 3b).

Autoinducer Assay. Autoinducers produced by V. cholerae N16961 cell cultures were measured using a Vibrio harveyi bioluminescence assay based on the one developed by Greenberg, et. al⁴⁵, and used extensively to study cross-species induction²⁹. Briefly, V. cholerae was grown in LB medium for 16 hours at 37° C. in the absence and presence of inhibitors as described in the previous paragraph. The cells were centrifuged at 13K rpm for 30 minutes, and the supernatant was filtered through a 0.2 μm sterile syringe filter. V. harveyi BB120 and BB170 were grown overnight in autobioinducer (AB) medium at 30° C., shaken at 225 rpm. The densely grown BB120 and BB170 cells were diluted 1:5000 in AB medium in a 96-well plate before addition of V. cholerae filtrate to 10% (v/v) of the total cell culture volume. This dilution prevents the V. harveyi cells from responding to their own autoinducers. The plates were incubated at 30° C., and luminescence was measured on a Promega Glomax luminometer. Maximum light response to exogenous AIs was observed after 4 hours of incubation, and was hence set as incubation time for all assays. AI background correction used sterile growth media treated as a sample and light output from this incubation was used as blank. The magnitude of induction is taken as the ratio of light output induced by the V. cholerae filtrate relative to blank, and was plotted against concentration of inhibitor, and fitted to the following hyperbolic equation using KaleidaGraph 3.6 to obtain the IC₅₀:

$$y = {y}_{0} - \frac{c [ I ]}{{IC}_{50} + [ I ]}$$

where y is the magnitude of induction at inhibitor concentration [I]; y₀ is magnitude of induction in the absence of inhibitor (untreated sample); c is the maximum difference between treated and untreated sample, and IC₅₀ is the inhibitor concentration representing half maximal induction. The average of at least six replicates was taken, with outliers greater than two standard deviations removed from analysis. A control experiment was included where dilute BB170 and BB120 were incubated with filtered supernatant of untreated V. cholerae cell culture containing inhibitors exogenously added at concentrations corresponding to the treatment conditions. This was done to rule out any effect the inhibitors might have on the AIs already secreted in the media and the latter's ability to induce bioluminescence in the reporter strains.

Autoinducer-2 production in wild-type E. coli, wild-type treated with inhibitor, and an MTAN knockout mutant was determined using the assay described above. The cells were grown in AB medium at 37° C. for 16 hours, and in the presence of 5-1000 nM BuT-DADMe-ImmA (for the wild-type E. coli). Cell free fluids were incubated with V. harveyi BB170, and bioluminescence was measured after incubation at 30° C. for 4 hours.

### Results

MTAN transition state analogues are picomolar inhibitors of VcMTAN. VcMTAN has a substrate specificity for hydrolysis of the N-glycosidic bonds of both MTA and SAH. It has a Kₘ of 3 μM for MTA and a k_{cat} of 2 s⁻¹. For SAH, the Kₘ and k_{cat} values are 24 μM, and 0.5 s⁻¹, respectively. With a k_{cat}/Kₘ ratio of 6.6×10⁵M⁻¹ s⁻¹ for MTA, VcMTAN's catalytic efficiency is 60-fold greater than the S. pneumoniae isoform, and 14-fold less than for E. coli MTAN^{23,25}. Dissociation constants of VcMTAN for the transition state analogues MT-, EtT-, and BuT-DADMe-ImmA are in the mid-picomolar range, compared to E. coli MTAN in the low picomolar, and to S. pneumoniae MTAN in the nanomolar ranges (Table 2)^{23,25}. Thus, VcMTAN is inhibited by transition state analogues with an affinity intermediate to that for E. coli and S. pneumoniae MTANs with the same transition state analogues, as predicted by the catalytic enhancement provided by the enzymes. Reaction progress curves in the presence of various concentrations of MT-, EtT-, and BuT-DADMe-ImmA revealed time-dependent, slow-onset inhibition, yielding overall dissociation constants of 73, 70, and 208 pM, respectively (FIG. 3a).

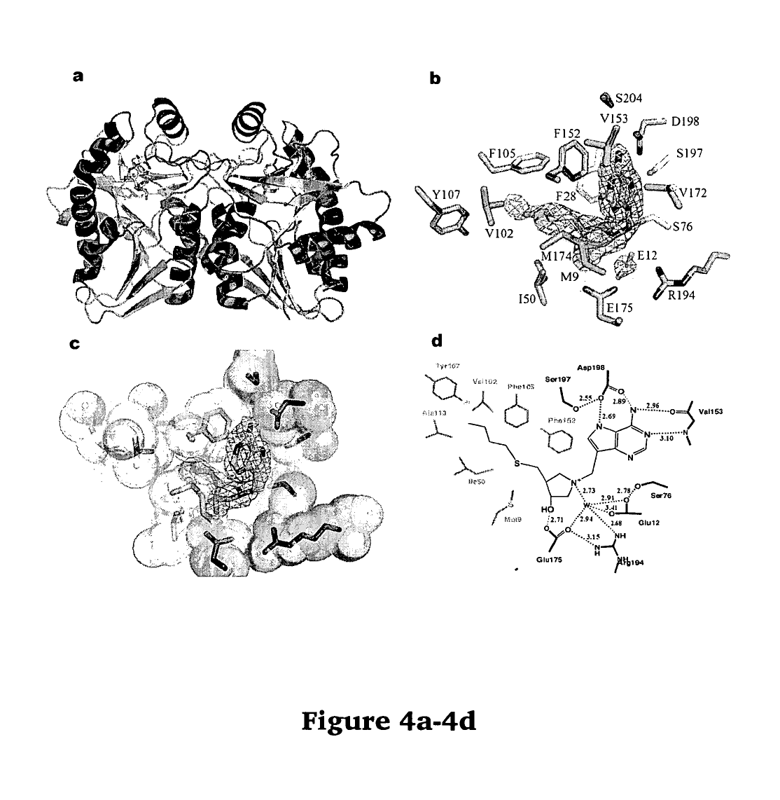

Crystal structure of VcMTAN-BuT-DADMe-ImmA complex. The crystal structure of VcMTAN in complex with BuT-DADMe-ImmA was determined to 2.3 Å resolution to define the determinants responsible for inhibitor binding (FIG. 4). The final atomic model contains residues 1-230 for each monomer of VcMTAN in the asymmetric unit. The largest part of the N-terminal 6-His tag and the last C-terminal residue, 231, were omitted from the structure model due to lack of electron density. The model exhibits good geometry, and the majority of the residues (89%) are located in the most favored region of the Ramachandran Plot. All remaining amino acids (11%) are in the allowed region (Table 1).

TABLE 2
Inhibition constants for purified MTAN activity, cellular MTAN activity, and
autoinducer (AI) production determined as described in the experimental section. The
structure of S-substituted DADMe-ImmucillinA is shown, along with MT—, EtT— and BuT—
substituents.

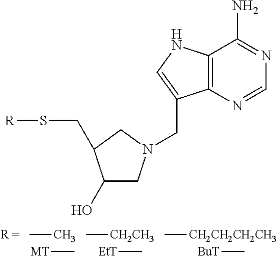

| | Purified enzyme | Cellular MTAN | AI Inhibition | |
|---|---|---|---|---|
| | inhibition | Inhibition | IC₅₀, nM | |
| R-group | Kᵢ*, pM | IC₅₀, nM | BB170 (ai¹⁻ai²⁺) | BB120 (ai¹⁺ai²⁺) |
| MT— | 73 ± 5 | 27 ± 4 | 0.94 ± 0.13 | 10.5 ± 2.6 |
| EtT— | 70 ± 4 | 31 ± 7 | 11.0 ± 2.0 | 14.0 ± 2.0 |
| BuT— | 208 ± 46 | 6 ± 1 | 1.4 ± 0.3 | 1.0 ± 0.2 |

The VcMTAN structure complexed with BuT-DADMe-ImmA has two monomers in the asymmetric unit related by 2-fold noncrystallographic symmetry which corresponds to the functional dimer (FIG. 4a). Density for the inhibitor in the active site was clearly visible at a σ-level of 5, in maps generated after the first round of refinement in REFMAC5 (FIG. 4b). The structure of the VcMTAN monomer is a single mixed α/β domain with central twisted nine-stranded mixed β-sheet surrounded by six α-helices (FIG. 4a). Both the monomeric structure and the dimeric form are very similar to the MTAN from E. coli with rms deviations of 0.44 Å comparing the Cα of the two structures although the sequence identity is only 59%²⁷. The dimer interface involves hydrophobic residues coming from two α-helices and three loop regions from each monomer.

The catalytic site is situated in a pocket formed by residues from β10, a loop between β8 and α4 and a loop contributed by the adjacent subunit (FIG. 4b,c). The catalytic site can be divided into three parts, the base binding site, the ribose binding site and the 5′-alkylthio-binding site. The purine base contacts Phe152, main chain atoms of Val153, and side chain of Asp198 (FIG. 4d). Phe152 makes hydrophobic stacking interactions with the 9-deazaadenine base of the inhibitor. The carbonyl oxygen of Val153 makes a potential hydrogen bond to N6 (2.95 Å) of the base while the amide nitrogen of Val153 makes a hydrogen bond to N1 (3.15 Å). The side chain of Asp198 interacts with hydrogen bonds to N6 (3.1 Å) and N7 (3.0 Å) of the base. Ser197 hydrogen bonds to OD2 (3.0 Å) of Asp198 and places the side chain in an orientation favorable for catalysis. Amide nitrogen of Val199 may also orient the Asp 198 for catalysis by hydrogen bonding to OD1 (3.2 Å) of the latter.

The pyrrolidine moiety participate in interactions with Met9, Phe208 and Met174 on both sides of the ribosyl mimic. The pyrrolidine moiety, which lacks the 2′ OH shares hydrogen bonds with Glu175 and the proposed catalytic water (WAT3) (FIG. 4d). The OE1 of Glu175 hydrogen bonds to the 3′-hydroxyl of the pyrrolidine with a distance of 2.8 Å. The protonated N1′ nitrogen of the pyrrolidine makes a potential hydrogen bond with WAT3 (2.8 Å). WAT3 is further stabilized by several hydrogen bonds from OE2 of Glu175 (2.9 Å), OE1 and OE2 of Glu12 (3.1 and 2.9 Å), and NH1 of Arg194 (2.7 Å). The side chain of Ser76 is also within hydrogen bond distance to OE2 of Glu12 (2.5 Å) and is involved in holding Glu12 in place for catalysis.

The 5′-butylthio group is surrounded by hydrophobic residues including Met9, Ile50, Val102, Phe105, Ala113, Phe152, Met174, Tyr107 and Phe208 (FIG. 4c). Both subunits form the catalytic site and Tyr107, Phe105, Ala 113 and Val102 reside on the adjacent subunit.

Inhibition of cellular MTAN activity. The presence of the transition state analogues had no effect on the growth of V. cholerae N16961 as demonstrated by the invariant OD₆₀₀ with inhibitor concentrations to 1 μM, up to 14,000 times the Kᵢ* value (FIG. 3d).

Inhibition of MTAN activity in cells was determined by culturing cells with inhibitors and assaying cleared lysates from washed cells with radiolabeled MTA. The activity of cell lysate from cells cultured without inhibitor was 89±3 pmol/min/OD₆₀₀ unit. This average was taken from each of the three inhibitor sets, and reflects the variability in the cell density attained by overnight cultures, and also in the amount of active MTAN in extracts. Extracts from cells grown in the presence of variable concentrations of transition state analogues showed dose-dependent inhibition of adenine conversion, giving IC₅₀ values for the loss of cellular MTAN activity of 27, 31, and 6 nM with MT-, EtT-, and BuT-DADMe-ImmA, respectively (Table 2 and FIG. 3b).

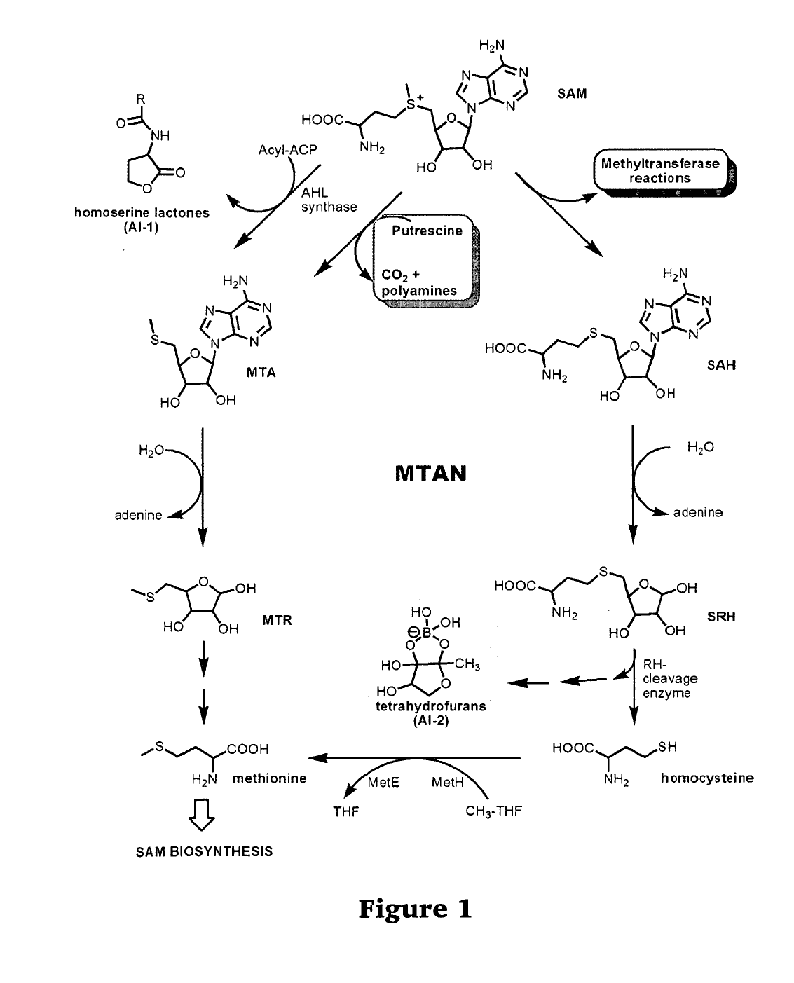

Inhibition of autoinducer production. Under the same conditions used to assay the inhibition of cellular MTAN activity, autoinducer production by V. cholerae N16961 was measured as a function of inhibitors (FIG. 1).

Luminescence from the actual samples compared to the blank medium was reported as the magnitude of induction, which reached 13.5 (±4.5) and 2.3 (±1.0) for quorum sensing reporter strains BB170 and BB120, respectively. V. harveyi BB170 responds to the presence of AI-2 alone, whereas BB120 responds to both AI-1 and AI-2. Inhibitors caused the AI response to become progressively weaker as inhibitor concentration increased, and was completely inhibited at 1 μM (FIG. 3c). Transition state analogues alone, at concentrations present in AI detection assays, had no effect on light output from the reporter strains. The IC₅₀ for suppression of light induction in BB170 was determined to be 0.94, 11, and 1.4 nM with MT-, EtT-, and BuT-DADMe-ImmA, whereas in BB120 the IC₅₀ inhibition constants were 10.5, 14, and 1 nM for the same inhibitors (Table 2).

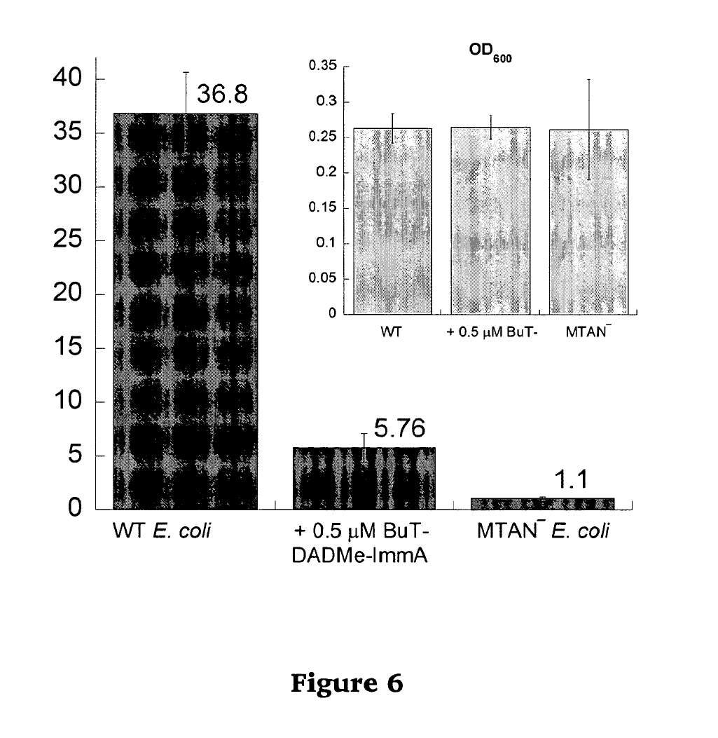

Autoinducer production in MTAN E. coli. Treated and untreated wild-type E. coli, as well as the MTAN knockout mutant grew in AB medium to similar OD₆₀₀ values (FIG. 6). AI induction in BB170 reached 37-fold for the wild-type compared to blank, while administration of BuT-DADMe-ImmA resulted in a dose-dependent inhibition of AI-2 induction with an IC₅₀ of 125±24 nM. At only four times this IC₅₀ value, induction was down to 6-fold (FIG. 6). The extent of AI-2 induction for the MTAN knockout was nearly nothing, suggesting that genetic ablation of MTAN in E. coli also inhibits synthesis of quorum sensing molecules.

### Discussion

MT-, EtT-, and BuT-DADMe-ImmA showed time-dependent, slow-onset inhibition of VcMTAN, with overall dissociation constants of 73, 70, and 208 pM, respectively. These are among the lowest dissociation constants for targets in quorum sensing pathways and are exceeded only by values from the same family of inhibitors with EcMTAN which are one to two orders of magnitude lower²³. Slow onset inhibition is typical for transition state analogues where binding to enzyme equilibrates the protein to a new conformation on the scale of seconds to minutes. The enzyme-inhibitor complex conformational change is characterized by a slow off rate that stabilizes the enzyme in its inhibited form. Kₘ/Kᵢ values for all three inhibitors are approximately 10⁴, showing strong preference for the transition state analogues over the substrate MTA.

The MTANs have dual substrate specificity for MTA and SAH, and are expected to accommodate both methylthio- and homocysteine groups in a manner proportional to their Kₘ values. Transition state analogues that differ only in their 5′-substituents permit direct comparison of VcMTAN's preference for these groups. MT- and EtT-groups are equally favored at this position, and are also equivalent in blocking quorum sensing in vitro. The dissociation constant increases three-fold however, in going from ethyl- to butyl-substituted DADMe-ImmA and suggests a modest size specificity within the 5′-binding pocket delineated by the 2-carbon difference of these groups.

Recently, a method for predicting the transition state structure for MTANs was reported, using dissociation constants for known transition state analogues²⁶. This method classifies MTANs as having either early or late dissociative transition states, depending on the ratio of its dissociation constants for ImmA and DADMe-ImmA, which are analogues of the early and late dissociative transition states, respectively. Dissociation constants were determined for VcMTAN with methylthio-, ethylthio-, benzylthio-, and para-chloro-phenylthio-ImmucillinA (data not shown). For the MT-ImmA/DADMe-ImmA inhibitor pair, VcMTAN gives a K_{ImmA}/K_{DADMe-ImmA} of 135, indicating a strong preference for the transition state analogue that resembles a late transition state. This analysis predicts a late dissociative transition state for VcMTAN, similar to that of E. coli and S. pneumoniae. In addition, not only were the ImmA dissociation constants much higher than their DADMe-ImmA counterparts for the four above-mentioned compounds, there was no slow onset phase in their inhibition profiles. Thus, the DADMe-ImmA compounds are better mimics of VcMTAN's transition state, and strongly suggests a late dissociative one.

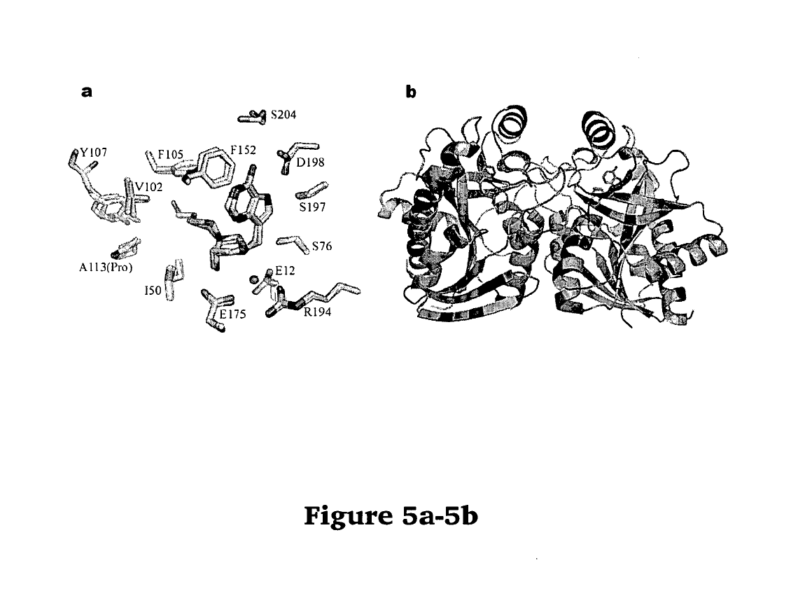

The crystal structure of BuT-DADMe-ImmA in complex with VcMTAN is similar to the crystal structure of EcMTAN in complex with MT-DADMe-ImmA (FIG. 5a)²⁷. The inhibitors in the two structures share a virtual overlap of the 9-deazaadenine and the pyrrolidine ribocation mimic. Similar to EcMTAN, tight binding in the VcMTAN complex is proposed to originate mainly from the conformation adopted by the pyrrolidine group of the inhibitor that allows for the cation at N1′ to be in close proximity to the putative water nucleophile which organizes the geometry of Ser76, Glu12, Arg194, and Glu175 around the catalytic site. The pKa of the N1′ pyrrolidine nitrogen is 8, making it cationic at physiological pH. The DADMe-ImmA inhibitors lack the 2′-hydroxyl moiety of ribosyl groups and allow the presumed catalytic water to be close to the N1′ with a distance of 2.7 Å. This distance was also found to be 2.6 Å in the case of the EcMTAN-MT-DADMe-ImmA structure²⁷. The affinity to EcMTAN for MT-DADMe-ImmA is similar to the affinity of VcMTAN for BuT-DADMe-ImmA. Based on the favorable hydrophobic interactions between the 5′-butylthio group and the hydrophobic pocket in the protein, additional binding affinity would be anticipated relative to MT-DADMe-ImmA. The 3-fold decrease in affinity for BuT-inhibitor relative to MT-inhibitor may correspond to the entropy loss upon binding the flexible butyl group at the catalytic site.

BuT-DADMe-ImmA binds 1000 times stronger to the EcMTAN than to the VcMTAN. Comparisons of the structures overall and the active sites do not reveal obvious explanations for the difference (FIG. 5a,b). The two structures share 59% sequence identity and have almost identical active sites. However, recent studies have demonstrated that residues remote from the active site of purine nucleoside phosphorylase contribute to transition state structure and catalytic efficiency through dynamic motion²⁸. The enhanced catalytic efficiency and inhibitor binding specificity of EcMTAN may also involve the full dynamic architecture of the protein.

Biological effectiveness of MTAN inhibitors in the context of the cell was measured in cell lysates of a virulent strain of Vibrio cholerae (N16961) grown in the presence of inhibitors. Growth profiles showed no difference in the absence or presence of inhibitors, demonstrating that the compounds are not toxic (FIG. 3d). MTAN activity as judged by direct assays, was inhibited in a dose-dependent manner, giving IC₅₀ values of 27, 31, and 6 nM for MT-, EtT-, and BuT-DADMe-ImmA, respectively. These results demonstrate cell permeability for these compounds, most notably in the case of BuT-DADMe-ImmA. Despite having a 3-fold lower affinity with purified VcMTAN, BuT-DADMe-ImmA inhibited cellular VcMTAN activity 5-fold better than its MT-, and EtT-counterparts (Table 2). Despite this advantage, BuT-DADMe-ImmA inhibition of VcMTAN activity in the cell requires a 30-fold increase above the Kᵢ*, suggesting a significant diffusion barrier. With MT-, and EtT-DADMe-ImmA, the diffusion barrier requires a gradient close to 500-fold to inhibit VcMTAN in growing cells.

Under the same conditions used in cellular MTAN inhibition, autoinducer production provides characterization of autoinducer sensitivity to MTAN inhibition. In the absence of inhibitors, V. cholerae cell media gave a 13-fold induction by BB170 response. In the presence of MT-, EtT-, and BuT-DADMe-ImmA light production was completely inhibited by nanomolar concentration of the inhibitors to give IC₅₀ values of 0.94, 11, and 1.4 nM. The potency of these compounds to inhibit purified and cellular VcMTAN translated to the inhibition of autoinducer-2 production in V. cholerae N16961.

Growth medium from V. cholerae cell culture induced luminescence in BB120 only 2-fold. Marginal induction in BB120 was previously observed by Bassler et. al. for other strains of V. cholerae subjected to the same assay²⁹. They postulated that in the presence of system 1 (response system for AI-1) in V. harveyi BB120 strain, system 2 (response system for AI-2) is less sensitive to induction²⁹. In the presence of MT, EtT-, and BuT-DADMe-ImmA, light-induction in BB120 was inhibited by MTAN inhibitors with IC₅₀ values of 10.5, 14 and 1 nM, respectively. MTAP inhibitors are powerful inhibitors of quorum sensing induction in both reporter strains. The inhibition constants for BB120 induction follow the same trend as the cellular MTAN inhibition by the three transition state analogues, with BuT-DADMe-ImmA being slightly more potent.

Control experiments established that exogenous inhibitors did not influence the luminescence data. Inhibitors in the culture media have no effect on the autoinducers already present, supporting action of the transition state analogues on MTAN of V. cholerae cells for their effect on autoinducer production.

Genetic interruption of MTAN is expected to inhibit autoinducer production if the MTAP inhibitors have their effect through this target. In E. coli, knocking out MTAN reduces AI-2 induction to almost undetectable levels without affecting growth (FIG. 6). Knocking out MTAN in E. coli, and inhibiting with a transition state analogue both resulted in a reduction of AI-2 production without killing the cells. Thus, MTAN activity is nonessential in these bacteria, and it plays an important role in autoinducer-2 production.

Transition state theory has had several recent successes in the development of powerful inhibitors with in vivo effects against target enzymes. MT-, EtT-, and BuT-DADMe-ImmA are transition state analogues of bacterial MTANs and they show high potency in disrupting quorum sensing molecules in Vibrio cholerae. Although V. cholerae is a valuable test organism for quorum sensing studies, mounting evidence suggests that disrupting quorum sensing in this pathogen may induce expression of virulence factors and promote biofilm formation³⁰⁻³². While Vibrio cholerae possesses a uniquely inverted quorum sensing mechanism to increase survival and infectivity, several other pathogens use quorum sensing of autoinducers to signal expression of virulence factors, colonization, and biofilm formation. Escherichia coli, Streptococcus pneumoniae, Neisseria meningitidis, Klebsiella pneumoniae, Staphylococcus aureus, Helicobacter pylori, are some of the most aggressive human pathogens, and published evidence supports quorum sensing as promoting pathogenesis in these species^{8,33-37}. All these bacterial species possess MTANs, and the transition state analogues described here are potent in inhibiting purified MTANs from these sources^{23,25,26}. The potential of inhibiting quorum sensing by targeting MTAN is expected to extend to all of these other pathogens.

### REFERENCES

1. Fuqua, W. C., Winans, S. C. & Greenberg, E. P. Quorum sensing in bacteria: the LuxR-LuxI family of cell density-responsive transcriptional regulators. J. Bacteriol. 176, 269-275 (1994).
2. Sperondio, V. Novel approaches to bacterial a infection therapy by interfering with bacteria-to-bacteria signaling. Expert Rev. Anti Infect. Ther. 5, 271-276 (2007).
3. Vendeville, A., Winzer, K., Heurlier, K., Tang, C. M. & Hardie, K. R. Making ‘sense’ of metabolism: Autoinducer-2, LuxS and pathogenic bacteria. Nat. Rev. Microbiol. 3, 383-396 (2005).
4. Cegelski, L., Marshall, G. R., Eldridge, G. R. & Hultgren, S. J. The biology and future prospects of antivirulence therapies. Nat. Rev. Microbiol. 6, 17-27 (2008).
5. Waters, C. M. & Bassler, B. L. Quorum sensing: Cell-to-cell communication in bacteria. Annu. Rev. Cell Dev. Biol. 21, 319-346 (2005).
6. Winzer, K. & Williams, P. Quorum sensing and the regulation of virulence gene expression in pathogenic bacteria. Int. J. Med. Microbiol. 291, 131-143 (2001).
7. Stroeher, U. H., Paton, A. W., Ogunniyi, A. D. & Paton, J. C. Mutation of luxS of Streptococcus pneumoniae affects virulence in a mouse model. Infect. Immun. 71, 3206-3212 (2003).
8. Winzer, K. et al. Role of Neisseria meningitidis luxS in cell-to-cell signaling and bacteremic infection. Infect. Immun. 70, 2245-2248 (2002).
9. Lesprit, P. et al. Role of the Quorum-sensing system in experimental pneumonia due to Pseudomonas aeruginosa in rats. Am. J. Respir. Crit. Care Med. 167, 1478-1482 (2003).
10. Parsek, M. R., Val, D. L., Hanzelka, B. L., Cronan, J. E. & Greenberg, E. P. Acyl homoserine-lactone quorum-sensing signal generation. Proc. Nat. Acad. Sci. USA 96, 4360-4365 (1999).
11. Harasawa, H. et al. Chemotherapy targeting methylthioadenosine phosphorylase (MTAP) deficiency in adult T cell leukemia (ATL). Leukemia 16, 1799-1807 (2002).
12. Basu, I. et al. A transition state analogue of 5′-methylthioadenosine phosphorylase induces apoptosis in head and neck cancers. J. Biol. Chem. 282, 21477-21486 (2007).
13. Singh, V., Lee, J. E., Nunez, S., Howell, P. L. & Schramm, V. L. Transition state structure of 5′-methylthioadenosine/S-adenosylhomocysteine nucleosidase from Escherichia coli and its similarity to transition state analogues. Biochemistry 44, 11647-11659 (2005).
14. Singh, V. & Schramm, V. L. Transition-state analysis of S-pneumoniae 5′-methylthioadenosine nucleosidase. J. Am. Chem. Soc. 129, 2783-2795 (2007).
15. Singh, V., Luo, M., Brown, R. L., Norris, G. E. & Schramm, V. L. Transition-state structure of Neisseria meningitides 5′-methylthioadenosine/S-adenosylhomocysteine nucleosidase. J. Am. Chem. Soc. 129, 13831-13833 (2007).
16. Singh, V. & Schramm, V. L. Transition-state structure of human 5′-methylthioadenosine phosphorylase. J. Am. Chem. Soc. 128, 14691-14696 (2006).
17. Balakrishnan, K., Nimmanapalli, R., Ravandi, F., Keating, M. J. & Gandhi, V. Forodesine, an inhibitor of purine nucleoside phosphorylase, induces apoptosis in chronic lymphocytic leukemia cells. Blood 108, 2392-2398 (2006).
18. Robak, T. L.-M., E.; Koerycka, A.; Robak, E. Purine nucleoside analogs as immunosuppressive and antineoplastic agents: mechanism of action and clinical activity. Curr. Med. Chem. 13, 3165-3189 (2006).
19. Galmarini, C. M. Drug evaluation: Forodesine—a PNP inhibitor for the treatment of leukemia, lymphoma and solid tumor. IDrugs 9, 712-722 (2006).
20. Schramm, V. L. Development of transition state analogues of purine nucleoside phosphorylase as anti-T-cell agents. Biochim. Biophys. Acta, Mol. Basis Dis. 1587, 107-117 (2002).
21. Evans, G. B., Furneaux, R. H., Schramm, V. L., Singh, V. & Tyler, P. C. Targeting the polyamine pathway with transition-state analogue inhibitors of 5′-methylthioadenosine phosphorylase. J. Med. Chem. 47, 3275-3281 (2004).
22. Evans, G. B. et al. Second generation transition state analogue inhibitors of human 5′-methylthioadenosine phosphorylase. J. Med. Chem. 48, 4679-4689 (2005).
23. Singh, V. et al. Femtomolar transition state analogue inhibitors of 5′-methylthioadenosine/S-adenosylhomocysteine nucleosidase from Escherichia coli. J. Biol. Chem. 280, 18265-18273 (2005).
24. Singh, V. et al. Picomolar transition state analogue inhibitors of human 5′-methylthioadenosine phosphorylase and x-ray structure with MT-Immucillin-A. Biochemistry 43, 9-18 (2004).
25. Singh, V. et al. Structure and inhibition of a quorum sensing target from Streptococcus pneumoniae. Biochemistry 45, 12929-12941 (2006).
26. Gutierrez, J. A. et al. Picomolar inhibitors as transition-state probes of 5′-methylthioadenosine nucleosidases. ACS Chem. Biol. 2, 725-734 (2007).
27. Lee, J. E. et al. Structural rationale for the affinity of pico- and femtomolar transition state analogues of Escherichia coli 5′-methylthioadenosine/S-adenosylhomocysteine nucleosidase. J. Biol. Chem. 280, 18274-18282 (2005).
28. Saen-Oon, S., Ghanem, M., Schramm, V. L. & Schwartz, S. D. Remote mutations and active site dynamics correlate with catalytic properties of purine nucleoside phosphorylase. Biophys. J. 94, 4078-4088 (2008).
29. Bassler, B. L., Greenberg, E. P. & Stevens, A. M. Cross-species induction of luminescence in the quorum-sensing bacterium Vibrio harveyi. J. Bacteriol. 179, 4043-4045 (1997).
30. Zhu, J. et al. Quorum-sensing regulators control virulence gene expression in Vibrio cholerae. Proc. Natl. Acad. Sci. U.S.A. 99, 3129-3134 (2002).
31. Matson, J. S., Withey, J. H. & DiRita, V. J. The regulatory network controlling Vibrio cholerae virulence gene expression. Infect. Immun., IAI.01094-07 (2007).
32. Waters, C. M., Lu, W., Rabinowitz, J. D. & Bassler, B. L. Quorum sensing controls biofilm formation in Vibrio cholerae through modulation of cyclic di-GMP levels and repression of vpsT. J. Bacteriol. 190, 2527-2536 (2008).
33. Surette, M. G. & Bassler, B. L. Quorum sensing in Escherichia coli and Salmonella typhimurium. Proc. Nat. Acad. Sci. USA 95, 7046-7050 (1998).
34. Dunny, G. M. & Leonard, B. A. B. Cell-cell communication in gram-positive bacteria. Annu. Rev. Microbiol. 51, 527-564 (1997).
35. Balestrino, D., Haagensen, J. A. J., Rich, C. & Forestier, C. Characterization of type 2 quorum sensing in Klebsiella pneumoniae and relationship with biofilm formation. J. Bacteriol. 187, 2870-2880 (2005).
36. Joyce, E. A. et al. LuxS Is required for persistent pneumococcal carriage and expression of virulence and biosynthesis genes. Infect. Immun. 72, 2964-2975 (2004).
37. Rader, B. A., Campagna, S. R., Semmelhack, M. F., Bassler, B. L. & Guillemin, K. The quorum-sensing molecule autoinducer 2 regulates motility and flagellar morphogenesis in Helicobacter pylori. J. Bacteriol. 189, 6109-6117 (2007).
38. Evans G B, F. R., Lenx D H, Painter G F, Schramm V L, Singh V, Tyler P C. Second generation transition state analogue inhibitors of human 5′-methylthioadenosine phosphorylase. J Med Chem 48, 4679-89 (2005).
39. Otwinowski Z, a. M. W. Processing of X-ray diffraction data collected in oscillation mode. Methods Enzymol 276, 307-326 (1997).
40. Potterton E, B., P., Turkenburg, M., and Dodson, E. A graphical user interface to the CCP4 program suite. Acta Cryst. D59, 1131-1137 (2003).
41. Vagin, A. & Teplyakov, A. MOLREP: an automated program for molecular replacement. J. Appl. Cryst. 30, 1022-1025 (1997).
42. Murshudov, G. N., Vagin, A. A. & Dodson, E. J. Refinement of macromolecular structures by the maximum-likelihood method. Acta Crystallogr., Sect. D: Biol. Crystallogr. 53, 240-255 (1997).
43. Emsley P, a. C., K. Model-building tools for molecular graphics. Acta Crystallogr. D Biol. Crystallogr 60, 2126-2132 (2004).
44. DeLano, W. L. The PyMOL molecular graphics system. (DeLano Scientific, Palo Alto, Calif., USA, 2002).
45. Greenberg, E. P., Hastings, J. W. & Ulitzur, S. Induction of luciferase synthesis in Beneckea harveyi by other marine bacteria. Arch. Microbiol. 120, 87-91 (1979).
46. Kamath, V. P., et al. Synthesis of a potent 5′-methylthioadenosine/S-adenosylhomocysteine (MTAN) inhibitor. Bioorganic & Medicinal Chemistry Letters. 16(10):2662-2665 (2006).

1. A method for treating a bacterial infection in a subject comprising administering to the subject a sub-growth inhibiting amount of a 5′-Methylthioadenosine/S-adenosyl homocysteine nucleosidase (MTAN) inhibitor effective to treat the bacterial infection in the subject.

2. The method of claim 1, wherein the MTAN inhibitor comprises a compound having formula (I): 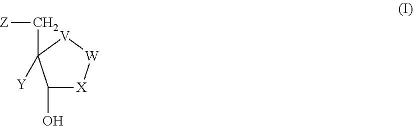
wherein:
V is selected from CH₂ and NH, and W is selected from NR¹ and NR²; or V is selected from NR¹ and NR², and W is selected from CH₂ and NH;
X is selected from CH₂ and CHOH in the R or S-configuration;
Y is selected from hydrogen, halogen and hydroxy, except where V is selected from NH, NR¹ and NR² then Y is hydrogen;
Z is selected from hydrogen, halogen, hydroxy, SQ, OQ and Q, where Q is an optionally substituted alkyl, aralkyl or aryl group;
R¹ is a radical of the formula (II) 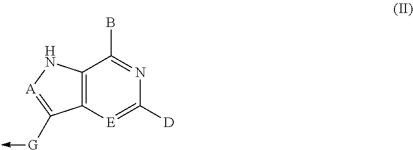
R² is a radical of the formula (III) 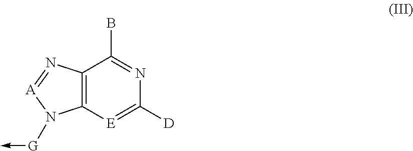
A is selected from N, CH and CR, where R is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups;
B is selected from OH, NH₂, NHR⁶, SH, hydrogen and halogen, where R⁶ is an optionally substituted alkyl, aralkyl or aryl group;
D is selected from OH, NH₂, NHR⁷, hydrogen, halogen and SCH₃, where R⁷ is an optionally substituted alkyl, aralkyl or aryl group;
E is selected from N and CH;
G is selected from CH₂ and NH, or G is absent, provided that where W is NR¹ or NR² and G is NH then V is CH₂, and provided that where V is NR¹ or NR² and G is NH then W is CH₂,
or a tautomer thereof, or a pharmaceutically acceptable salt thereof, or an ester thereof, or a prodrug thereof.

3. The method of claim 2, wherein Z is selected from hydrogen, halogen, hydroxy, SQ and OQ.

4. The method of claim 2, wherein V is CH₂.

5. The method of claim 2, wherein X is CH₂.

6. The method of claim 2, wherein G is CH₂.

7. The method of claim 2, wherein Z is OH.

8. The method of claim 2, wherein Z is SQ.

9. The method of claim 2, wherein where Z is Q.

10. The method of claim 2, wherein W is NR¹.

11. The method of claim 2, wherein W is NR².

12. The method of claim 2, wherein W is selected from NH, NR¹ or NR² and X is CH₂.

13. The method of claim 2, wherein V, X and G are all CH₂, Z is OH and W is NR¹.

14. The method of claim 2, wherein V, X and G are all CH₂, Z is SQ and W is NR¹.

15. The method of claim 2, wherein Q is aryl.

16. The method of claim 15, wherein Q is methyl, ethyl or butyl.

17. The method of claim 2, wherein Y is hydrogen.

18. The method of claim 2, wherein Y is hydroxy.

19. The method of claim 2, wherein B is hydroxy.

20. The method of claim 2, wherein B is NH₂.

21. The method of claim 2, wherein A is CH.

22. The method of claim 2, wherein A is N.

23. The method of claim 2, wherein D is H.

24. The method of claim 2, wherein D is NH₂.

25. The method of claim 2, wherein E is N.

26. The method of claim 2, wherein the MTAN inhibitor is selected from the group consisting of:
(3R,4S)-1-[(9-deazaadenin-9-yl)methyl]-3-hydroxy-4-(methylthiomethyl)pyrrolidine;
(3R,4S)-1-[(9-deazaadenin-9-yl)methyl]-3-hydroxy-4-(benzylthiomethyl)pyrrolidine;
(3R,4S)-1-[(8-Aza-deazaadenin-9-yl)methyl]-3-hydroxy-4-(benzylthiomethyl)pyrrolidine hydrochloride;
(3R,4S)-1-[(9-deazaadenin-9-yl)methyl]-3-hydroxy-4-(4-chlorophenylthiomethyl)pyrrolidine; and
(3R,4S)-1-[(9-deazaadenin-9-yl)methyl]-3-hydroxy-4-(2-phenylethyl)pyrrolidine hydrochloride.

27. The method of claim 1, wherein the MTAN inhibitor comprises a compound having formula (IV): 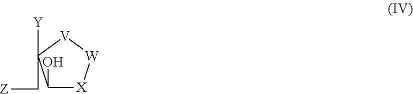
wherein:
V is selected from CH₂ and NH, and W is selected from NR¹ and NR²; or V is selected from NR¹ and NR², and W is selected from CH₂ and NH;
X is selected from CH₂ and CHOH in the R or S-configuration;
Y is selected from hydrogen, halogen and hydroxy, except where V is selected from NH, NR¹ and NR² then Y is hydrogen;
Z is selected from hydrogen, halogen, hydroxy, SQ, OQ and Q, where Q is an optionally substituted alkyl, aralkyl or aryl group;
R¹ is a radical of the formula (V) 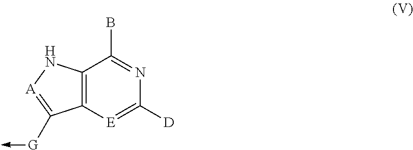
R² is a radical of the formula (VI) 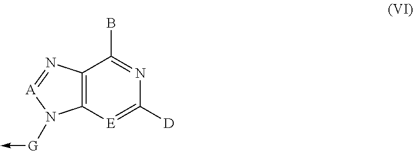
A is selected from N, CH and CR, where R is selected from halogen, optionally substituted alkyl, aralkyl or aryl, OH, NH₂, NHR³, NR³R⁴ and SR⁵, where R³, R⁴ and R⁵ are each optionally substituted alkyl, aralkyl or aryl groups;
B is selected from OH, NH₂, NHR⁶, SH, hydrogen and halogen, where R⁶ is an optionally substituted alkyl, aralkyl or aryl group;
D is selected from OH, NH₂, NHR⁷, hydrogen, halogen and SCH₃, where R⁷ is an optionally substituted alkyl, aralkyl or aryl group;
E is selected from N and CH;
G is selected from CH₂ and NH, or G is absent, provided that where W is NR¹ or NR² and G is NH then V is CH₂, and provided that where V is NR¹ or NR² and G is NH then W is CH₂;
or a tautomer thereof, or a pharmaceutically acceptable salt thereof, or an ester thereof, or a prodrug thereof.

28-53. (canceled)

54. The method of claim 1, wherein the MTAN inhibitor comprises a compound having formula (VII): 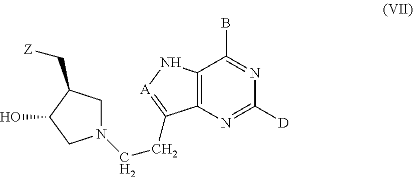
wherein:
A is N or CH;
B is OH or NH₂;
D is H, OH, NH₂ or SCH₃; and
Z is OH or SQ, where Q is an optionally substituted alkyl, aralkyl, or aryl group;
or a tautomer thereof; or a pharmaceutically acceptable salt thereof; or an ester prodrug form thereof.

55-65. (canceled)

66. The method of claim 1, wherein the MTAN inhibitor comprises a compound having formula (VIII): 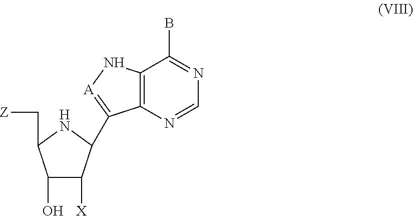
wherein:
A is selected from N, CH and CR,
where R is selected from halogen, optionally substituted alkyl, aralkyl and aryl, OH, NH₂, NHR¹, NR¹R² and SR³,
where R¹, R² and R³ are each optionally substituted alkyl, aralkyl or aryl groups;
B is selected from NH₂ and NHR⁴,
where R⁴ is an optionally substituted alkyl, aralkyl or aryl group;
X is selected from H, OH and halogen; and
Z is selected from H, Q, SQ and OQ,
where Q is an optionally substituted alkyl, aralkyl or aryl group;
or a tautomer thereof; or a pharmaceutically acceptable salt thereof; or an ester thereof; or a prodrug thereof;
with the proviso that the stereochemistry of the aza-sugar moiety is D-ribo or 2′-deoxy-D-erythro-.

67-80. (canceled)

81. The method of claim 1, wherein the MTAN inhibitor comprises a compound having formula (IX): 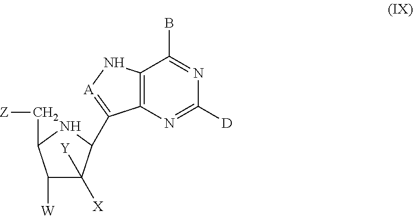
wherein:
A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups;
B is selected from OH, NH₂, NHR⁴, H and halogen, where R⁴ is an optionally substituted alkyl, aralkyl or aryl group;
D is selected from OH, NH₂, NHR⁵, H, halogen and SCH₃, where R⁵ is an optionally substituted alkyl, aralkyl or aryl group;
X and Y are independently selected from H, OH and halogen, with the proviso that when one of X and Y is hydroxy or halogen, the other is hydrogen;
Z is OH, or, when X is hydroxy, Z is selected from hydrogen, halogen, hydroxy, SQ and OQ, where Q is an optionally substituted alkyl, aralkyl or aryl group; and
W is OH or H, with the proviso that when W is OH, then A is CR where R is as defined above;
or a tautomer thereof; or a pharmaceutically acceptable salt thereof; or an ester thereof; or a prodrug thereof.

82-89. (canceled)

90. The method of claim 1, wherein the MTAN inhibitor comprises a compound having formula (X): 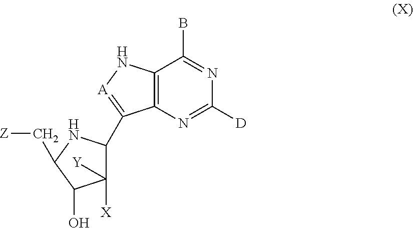
wherein A is CH or N;
B is chosen from OH, NH₂, NHR, H or halogen;
D is chosen from OH, NH₂, NHR, H, halogen or SCH₃;
R is an optionally substituted alkyl, aralkyl or aryl group; and
X and Y are independently selected from H, OH or halogen except that when one of X and Y is hydroxy or halogen, the other is hydrogen; and
Z is OH or, when X is hydroxy, Z is selected from hydrogen, halogen, hydroxy, SQ or OQ where Q is an optionally substituted alkyl, aralkyl or aryl group; or a tautomer thereof;
or a pharmaceutically acceptable salt thereof; or an ester thereof; or a prodrug thereof.

91-95. (canceled)

96. The method of claim 1, wherein the MTAN inhibitor comprises a compound having formula (XI): 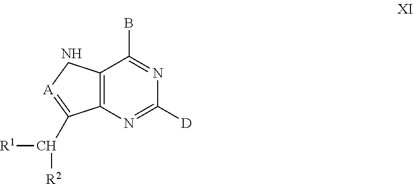
wherein:
R¹ is H or NR³R⁴;
R² is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, amino, hydroxy, or alkoxy groups; provided that when R¹ is H, R² is an alkyl, alkenyl, alkynyl, aralkyl, aralkenyl, aralkynyl, or aryl group which is substituted with at least one NR³R⁴ group;
R³ and R⁴, independently of each other, is H or is an alkyl, alkenyl, alkynyl, aralkyl, aralkenyl, aralkynyl, or aryl group each of which is optionally substituted with one or more hydroxy, alkoxy, thiol, alkylthio, arylthio, aralkylthio, halogen, carboxylic acid, carboxylate alkyl ester, nitro, or NR³R⁴ groups, where each alkylthio, arylthio and aralkylthio group is optionally substituted with one or more alkyl, halogen, hydroxy, or alkoxy groups;
A is N or CH;
B is NH₂ or NHR⁵,
R⁵ is an alkyl, alkenyl, alkynyl, aralkyl, aralkenyl, aralkynyl, or aryl group, each of which is optionally substituted with one or more halogen or hydroxy groups; and
D is H, OH, NH₂, or SCH₃; or a tautomer thereof, or a pharmaceutically acceptable salt thereof, or an ester prodrug form thereof.

97-133. (canceled)

134. The method of claim 1, wherein the MTAN inhibitor comprises a compound having formula (XII): 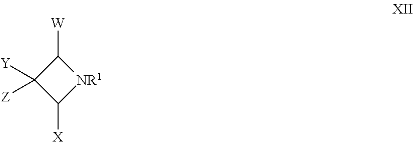
wherein:
W and X are each independently selected from hydrogen, CH₂OH, CH₂OQ and CH₂SQ;
Y and Z are each independently selected from hydrogen, halogen, CH₂OH, CH₂OQ, CH₂SQ, SQ, OQ and Q;
Q is an alkyl, aralkyl or aryl group each of which may be optionally substituted with one or more substituents selected from hydroxy, halogen, methoxy, amino, or carboxy;
R¹ is a radical of the formula (XIII) 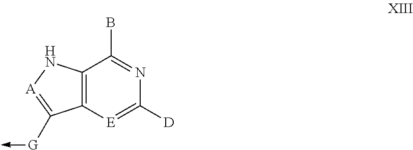
or R¹ is a radical of the formula (XIV) 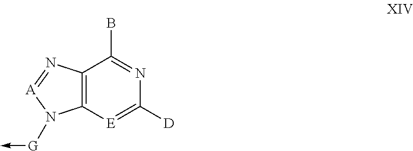
A is selected from N, CH and CR², where R² is selected from halogen, alkyl, aralkyl, aryl, OH, NH₂, NHR³, NR³R⁴ and SR⁵, where R³, R⁴ and R⁵ are each alkyl, aralkyl 5 or aryl groups optionally substituted with hydroxy or halogen, and where R² is optionally substituted with hydroxy or halogen when R² is alkyl, aralkyl or aryl;
B is selected from hydroxy, NH₂, NHR⁶, SH, hydrogen and halogen, where R⁶ is an alkyl, aralkyl or aryl group optionally substituted with hydroxy or halogen;
D is selected from hydroxy, NH₂, NHR⁷, hydrogen, halogen and SCH₃, where R⁷ is an alkyl, aralkyl or aryl. group optionally substituted with hydroxy or halogen;
E is selected from N and CH;
G is a C₁₋₄ saturated or unsaturated alkyl group optionally substituted with hydroxy or halogen, or G is absent; or a tautomer thereof, or a pharmaceutically acceptable salt thereof, or an ester thereof, or a prodrug thereof.

135-157. (canceled)

158. The method of claim 1, wherein the MTAN inhibitor is 5′-methylthio-(MT-) DADMe-ImmucillinA.

159. The method of claim 1, wherein the MTAN inhibitor is 5′-ethylthio-(MT-) DADMe-ImmucillinA.

160. The method of claim 1, wherein the MTAN inhibitor is 5′-butylthio-(MT-) DADMe-ImmucillinA.

161. The method of claim 1, wherein the sub-growth inhibiting amount of MTAN inhibitor inhibits quorum sensing in the bacteria.

162. The method of claim 1, wherein the bacterial infection is caused by Escherichia coli, Streptococcus pneumoniae, Pseudomonas aeruginosa, Neisseria meningitidis, Klebsiella pneumoniae, Staphylococcus aureus, or Helicobacter pylori.

163. A pharmaceutical composition comprising a sub-bacterial-growth inhibiting amount of a 5′-Methylthioadenosine/S-adenosyl homocysteine nucleosidase (MTAN) inhibitor and a pharmaceutically acceptable carrier.

164-167. (canceled)
